(19) United States
(12) Patent Application Publication
(10) Pub. No.: US 2025/0230439 A1
(43) Pub. Date: Jul. 17, 2025

(54) Methods For The Treatment Of ANGPTL3-Related Diseases And Disorders

(71) Applicant: Arrowhead Pharmaceuticals, Inc., Pasadena, CA (US)

(72) Inventors: Javier San Martin, Venice, CA (US); Thomas Schluep, Glendale, CA (US); James C. Hamilton, Sierra Madre, CA (US); Stacey Melquist, Pasadena, CA (US)

(21) Appl. No.: 19/059,378

(22) Filed: Feb. 21, 2025

Related U.S. Application Data
(63) Continuation of application No. PCT/US23/72573, filed on Aug. 21, 2023.
(60) Provisional application No. 63/373,175, filed on Aug. 22, 2022.
(60) Provisional application No. 63/376,302, filed on Sep. 20, 2022.
(60) Provisional application No. 63/382,916, filed on Nov. 9, 2022.
(60) Provisional application No. 63/492,548, filed on Mar. 28, 2023.
(60) Provisional application No. 63/506,910, filed on Jun. 8, 2023.

(51) Int. Cl. C12N 15/113; A61P 3/06

(52) U.S. Cl. CPC C12N 15/113; A61P 3/06; C12N 2310/14; C12N 2310/322; C12N 2310/351

(57) ABSTRACT

Described are methods for treating diseases and disorders that can be mediated in part by a reduction in ANGPTL3 gene expression in a human subject in need of treatment, using pharmaceutical compositions that include ANGPTL3 RNAi agents. The disclosed pharmaceutical compositions that include ANGPTL3 RNAi agents, when administered to a human subject in need thereof according to the methods disclosed herein, treat diseases and disorders associated with elevated triglyceride (TG) levels and/or elevated low density lipoprotein cholesterol (LDL-C), for example, hypertriglyceridemia (including severe hypertriglyceridemia (SHTG)), homozygous familial hypercholesterolemia (HoFH), hypertriglyceridemia induced pancreatitis, metabolic syndrome, obesity, hyperlipidemia, mixed dyslipidemia, abnormal lipid and/or cholesterol metabolism, atherosclerosis, cardiovascular disease, type II diabetes mellitus, coronary artery disease, non-alcoholic steatohepatitis, non-alcoholic fatty liver disease, heterozygous familial hypercholesterolemia (HeFH), statin resistant hypercholesterolemia, other dyslipidemias, and other metabolic-related disorders and diseases.

### CROSS REFERENCE TO RELATED APPLICATIONS

This application is a continuation application of International Application No. PCT/US2023/72573, filed on Aug. 21, 2023, which claims the benefit of priority of U.S. Provisional Patent Application Ser. No. 63/373,175, filed on Aug. 22, 2022, and U.S. Provisional Patent Application Ser. No. 63/376,302, filed on Sep. 20, 2022, and U.S. Provisional Patent Application Ser. No. 63/382,916, filed on Nov. 9, 2022, and U.S. Provisional Patent Application Ser. No. 63/492,548, filed on Mar. 28, 2023, and U.S. Provisional Patent Application Ser. No. 63/506,910, filed on Jun. 8, 2023, the contents of each of which are incorporated herein by reference in their entirety.

### SEQUENCE LISTING

This application contains a Sequence Listing which has been submitted in XML format and is hereby incorporated by reference in its entirety. The XML copy is named 30709-WO_SeqListing.xml, created Aug. 11, 2023, and is 40 kb in size.

### FIELD OF INVENTION

Disclosed herein are methods for the treatment of diseases and disorders that can be mediated at least in part by the silencing or inhibition of Angiopoietin-like 3 gene expression such as mixed dyslipidemia, familial hypercholesterolemia (HoFH), heterozygous familial hypercholesterolemia (HeFH), or atherosclerotic cardiovascular disease (ASCVD), using pharmaceutical compositions that include RNA interference (RNAi) agents that inhibit ANGPTL3 gene expression.

### BACKGROUND

Angiopoietin-like 3 (also called ANGPTL3, ANGPL3, ANG3, or angiopoietin-like protein 3) is an angiopoietin protein encoded by the human angiopoietin-like 3 gene that is reported to be involved in regulating lipid metabolism. ANGPTL3 is a 460-amino acid polypeptide that consists of a signal peptide, N-terminal coiled-coil domain, and a C-terminal fibrinogen (FBN)-like domain. ANGPTL3 is known to be primarily produced in hepatocytes in humans, and after synthesis is secreted into circulation. ANGPTL3 acts as an inhibitor of lipoprotein lipase, which catalyzes hydrolysis of triglycerides, and endothelial lipase, which hydrolyzes lipoprotein phospholipids. Inhibition of these enzymes can cause increases in plasma levels of triglycerides (TG), high-density lipoprotein cholesterol (HDL-C), and phospholipids. Further, loss-of-function mutations in ANGPTL3 lead to familial hypobetalipoproteinemia, which is characterized by low levels of triglycerides and low-density lipoprotein cholesterol (LDL-C) in plasma. In humans, loss-of-function in ANGPTL3 is also correlated with a decreased risk of atherosclerotic cardiovascular disease.

Given ANGPTL3's inhibitory role of various lipoproteins and thus triglycerides, reduced expression and reduced circulating levels of ANGPTL3 would be expected to increase clearance of LDL-C. HDL-C and TGs. It has been reported that individuals with ANGPTL3 loss-of-function mutations from birth present with very low levels of TGs, LDL-C and HDL-C (see, e.g., Minicocci et al., Mutations in the ANGPTL3 gene and familial combined hypolipidemia: a clinical and biochemical characterization, 97 J. Clin. Endocrinol Metab., E1266-75 (2012): Musunuru et al., Clinical characteristics and plasma lipids in subjects with familial combined hypolipidemia: a pooled analysis, 54 J. Lipid Res. 3481-90 (2010): Romeo et al., Rare loss-of-function mutations in ANGPTL family members contribute to plasma triglyceride levels in humans, 119 J. Clin. Invest. 70-79 (2009)). Patients with compound heterozygous or homozygous loss-of-function mutations can have undetectable serum ANGPTL3 with reductions in LDL-C of >65%, reductions in TG by >70%, and reductions in HDL-C by approximately 40% when compared to controls (Minicocci et al., Clinical characteristics and plasma lipids in subjects with familial combined hypolipidemia: a pooled analysis, 54 Lipid Res. 3481-90) (2013)). It has also been reported that individuals heterozygous for ANGPTL3 loss-of-function mutations demonstrate reduced LDL-C. HDL-C. TGs as well as reduction in the odds of developing atherosclerotic cardiovascular disease. The genetic validation consisting of low LDL-C and TGs coupled with reduced cardiovascular disease risk in ANGPTL3 deficient patients and proposed mechanism for these metabolic findings has promoted interest in methods capable of suppressing ANGPTL3.

A safe and effective therapeutic that targets ANGPTL3 may provide a beneficial impact in the treatment (including prophylactic treatment) of cardiometabolic diseases such as hypertriglyceridemia (including severe hypertriglyceridemia (SHTG)), obesity, hyperlipidemia, abnormal lipid and/or cholesterol metabolism, mixed dyslipidemia, atherosclerosis, type II diabetes mellitus, cardiovascular disease, coronary artery disease, non-alcoholic steatohepatitis, non-alcoholic fatty liver disease, homozygous familial hypercholesterolemia (HoFH), heterozygous familial hypercholesterolemia (HeFH), statin resistant hypercholesterolemia, and other metabolic-related disorders and diseases.

Currently, hypertriglyceridemia is often treated with one or more of niacin, fibrates, statins, and fish oil in moderate cases: however, in most cases, the reduction in serum TG is modest. Additionally, currently available therapeutics are often ineffective in patients with monogenic causes of very severe hypertriglyceridemia (such as patients with familial chylomicronemia syndrome (FCS)) because the majority of disease-causing mutations are in lipoprotein lipase (LPL), with mutations in cofactors or LPL interacting proteins apolipoprotein C-II (APO (2), apolipoprotein AV (APOA5), lipase maturation factor 1 (LMF1), and glycosylphosphatidylinositol anchored high density lipoprotein binding protein 1 (GPIHBP1) observed less frequently. These mutations lead to dysfunctional lipoprotein lipase, but functional lipoprotein lipase is required for optimal response to standard therapies.

Other attempts to inhibit ANGPTL3 have previously failed in clinical trials. For example, in January 2022 Pfizer Inc. and Ionis Pharmaceuticals, Inc. announced the discontinuation of an investigational antisense oligonucleotide (ASO) targeting ANGPTL3 that was being evaluated for potential indications in cardiovascular risk reduction and severe hypertriglyceridemia after a review of data from Phase 2b studies. (https://www.pfizer.com/news/press-release/press-release-detail/pfizer-and-ionis-announce-discontinuation-vupanorsen (last visited Aug. 22, 2022)) More specifically, not only did the Sponsors indicate that the magnitude of non-HDL-C and TG reduction not support further continuation of the program, but additionally dose-dependent increases in liver fat and higher doses were associated with elevations in the liver enzymes alanine aminotransferase (ALT) and aspartate aminotransferase (AST) led to the decision to discontinue further clinical studies with the ANGPTL3 ASO inhibitor.

Further, while evaluations with evinacumab, a monoclonal antibody targeting circulating ANGPTL3 have reported relatively potent reductions in LDL-C, HDL-C and TGs, an antibody approach would miss intra-hepatocyte ANGPTL3 which may be important for improvement of intra-hepatocyte triglyceride accumulation and insulin resistance (see, e.g., Graham et al., Cardiovascular and Metabolic Effects of ANGPTL3 Antisense Oligonucleotides, 377 N. Engl. J. Med. 222-32 (2017)). An antibody approach also requires monthly intravenous (IV) infusion administration (evkeeza.com), which may be inconvenient for patients and could impact adherence to treatment.

### SUMMARY

Described herein are methods of treating ANGPTL3-related diseases and disorders in a human subject in need thereof, the methods comprising administering to the human subject a pharmaceutical composition that includes the composition described in Table 2 (i.e., ANGPTL3 RNAi Drug Substance, also referred to herein as ADS-003) at a dose of at least about 50 mg of the ANGPTL3 RNAi Drug Substance, wherein the pharmaceutical composition is administered subcutaneously. In some embodiments, the pharmaceutical composition used in the methods disclosed herein comprises, consists of, or consists essentially of the Formulated ANGPTL3 RNAi Drug Substance as described in Table 3 (also referred to herein as ADS-003-1).

The ANGPTL3 RNAi Drug Substance described herein provided substantial potency and duration of effect while also not leading to significant increases in (i) liver fat, (ii) ALT, and (iii) AST. As shown in the data from the Phase 2b ARCHES-2 human clinical study presented herein (NCT04832971), the ANGPTL3 RNAi Drug Substance embodiment in doses of 50 mg, 100 mg, or 200 mg, provided durable inhibition of ANGPTL3 gene expression that resulted substantial median reductions in up to 59% in triglycerides (TG), and mean reductions in LDL-C(by up to 32%), non-HDL-C(by up to 36%), Apolipoprotein B (ApoB) (by up to 22%), remnant cholesterol (by up to 47%), and HDL-C(by up to 31%), while maintaining a favorable safety profile. Importantly, no statistical increase in liver fat was observed, and no significant increases in ALT or AST were observed.

The methods of treatment described herein lead to a reduction in ANGPTL3 expression in the human subject, which thereby results in a reduction of, among other things, serum triglyceride (TG) levels in the subject.

Additionally, described herein are methods of treating an ANGPTL3-related disease or disorder in a human subject in need thereof, the methods comprising administering to the human subject a pharmaceutical composition that includes the ANGPTL3 RNAi Drug Substance as described in Table 2 (i.e., ADS-003) at a dose of between about 50 mg and about 400 mg, wherein the pharmaceutical composition is administered subcutaneously and there is about one month between dose administrations (i.e., monthly dosing).

Further described herein are methods of treating an ANGPTL3-related disease or disorder in a human subject in need thereof, the methods comprising administering to the human subject a pharmaceutical composition that includes the ANGPTL3 RNAi Drug Substance as described in Table 2 at a dose of between about 50 mg and about 400 mg, wherein the pharmaceutical composition is administered subcutaneously and there is about 12 weeks or about three months between dose administrations (e.g., quarterly dosing or dosing every 12 weeks (q12w)).

Further described herein are methods of treating an ANGPTL3-related disease or disorder in a human subject in need thereof, the methods comprising administering to the human subject a pharmaceutical composition that includes the ANGPTL3 RNAi Drug Substance as described in Table 2 at a dose of between about 50 mg and about 400 mg, wherein the pharmaceutical composition is administered subcutaneously and there is about four months between dose administrations.

Further described herein are methods of treating an ANGPTL3-related disease or disorder in a human subject in need thereof, the methods comprising administering to the human subject a pharmaceutical composition that includes the ANGPTL3 RNAi Drug Substance as described in Table 2 at a dose of between about 50 mg and about 400 mg, wherein the pharmaceutical composition is administered subcutaneously and there is about six months between dose administrations (e.g., semi-annual or q6m dosing).

Also described herein are methods of treating an ANGPTL3-related disease or disorder in a human subject in need thereof, the methods comprising administering to the human subject a pharmaceutical composition that includes the ANGPTL3 RNAi Drug Substance as described in Table 2 at a dose of between about 50 mg and about 400 mg, wherein the pharmaceutical composition is administered subcutaneously, and wherein the initial dose is followed by a second dose about one month later, and thereafter for subsequent doses there is about three months between dose administrations.

Also described herein are methods of treating an ANGPTL3-related disease or disorder in a human subject in need thereof, the methods comprising administering to the human subject a pharmaceutical composition that includes the ANGPTL3 RNAi Drug Substance as described in Table 2 at a dose of between about 50 mg and about 400 mg, wherein the pharmaceutical composition is administered subcutaneously, and wherein the initial dose is followed by a second dose about one month later, and thereafter for subsequent doses there is about four months between dose administrations.

Described herein are methods of treating an ANGPTL3-related disease or disorder in a human subject in need thereof, the methods comprising administering to the human subject a pharmaceutical composition that includes the ANGPTL3 RNAi Drug Substance as described in Table 2 at a dose of between about 50 mg and about 400 mg, wherein the pharmaceutical composition is administered subcutaneously, and wherein the initial dose is followed by a second dose about one month later, and thereafter for subsequent doses there is about six months between dose administrations.

In some embodiments, the dose of ANGPTL3 RNAi Drug Substance administered in each dose is between about 50 mg and about 400 mg. In some embodiments, the dose of ANGPTL3 RNAi Drug Substance administered in each dose is between about 100 mg and about 300 mg. In some embodiments, the dose of ANGPTL3 RNAi Drug Substance administered in each dose is between about 200 mg and about 300 mg. In some embodiments, the dose of ANGPTL3 RNAi Drug Substance administered in each dose is at least about 50 mg. In some embodiments, the dose of ANGPTL3 RNAi Drug Substance administered in each dose is about 50 mg. In some embodiments, the dose of ANGPTL3 RNAi Drug Substance administered in each dose is about 100 mg. In some embodiments, the dose of ANGPTL3 RNAi Drug Substance administered in each dose is about 200 mg. In some embodiments, the dose of ANGPTL3 RNAi Drug Substance administered in each dose is about 300 mg. In some embodiments, the dose of ANGPTL3 RNAi Drug Substance administered in each dose is no greater than 400 mg. In some embodiments, the dose of ANGPTL3 RNAi Drug Substance administered in each dose is no greater than 300 mg. In some embodiments, the dose of ANGPTL3 RNAi Drug Substance administered in each dose is no greater than 200 mg.

The treatment methods disclosed herein can provide a substantial TG lowering effect for the treatment of ANGPTL3-related diseases and disorders such as hypertriglyceridemia (including severe hypertriglyceridemia (SHTG)), homozygous familial hypercholesterolemia (HoFH), hypertriglyceridemia induced pancreatitis, metabolic syndrome, obesity, hyperlipidemia, mixed dyslipidemia, abnormal lipid and/or cholesterol metabolism, atherosclerosis, cardiovascular disease, type II diabetes mellitus, coronary artery disease, non-alcoholic steatohepatitis, non-alcoholic fatty liver disease, heterozygous familial hypercholesterolemia (HeFH), statin resistant hypercholesterolemia, other dyslipidemias, and other metabolic-related disorders and diseases.

The methods disclosed herein can, in some embodiments, treat the ANGPTL3-related diseases or disorders by substantially lower TG levels and/or cholesterol levels and/or other lipid parameters, and thereby reducing the risk of developing hypertriglyceridemia (including severe hypertriglyceridemia (SHTG)), homozygous familial hypercholesterolemia (HoFH), hypertriglyceridemia induced pancreatitis, metabolic syndrome, obesity, hyperlipidemia, mixed dyslipidemia, abnormal lipid and/or cholesterol metabolism, atherosclerosis, cardiovascular disease, type II diabetes mellitus, coronary artery disease, non-alcoholic steatohepatitis, non-alcoholic fatty liver disease, heterozygous familial hypercholesterolemia (HeFH), statin resistant hypercholesterolemia, other dyslipidemias, and other metabolic-related disorders and diseases.

The pharmaceutical compositions that include ANGPTL3 RNAi agents disclosed herein can be administered to a human subject to inhibit the expression of an ANGPTL3 gene in the subject. In some embodiments, the subject is a human that has been previously diagnosed with having elevated triglyceride levels, over-expression of ANGPTL3 protein, or one or more ANGPTL3-related diseases or disorders.

Other objects, features, aspects, and advantages of the invention will be apparent from the following detailed description, accompanying figures, and from the claims.

### BRIEF DESCRIPTION OF THE DRAWINGS

FIG. 1A to 1D. Chemical structure representation of ANGPTL3 RNAi Drug Substance described in Table 2 (referred to herein as ADS-003: i.e., ANGPTL3 RNAi agent conjugated to a tridentate N-acetyl-galactosamine targeting group at the 5′ terminal end of the sense strand), shown in a sodium salt form. The sodium atoms are shown in FIGS. 1A to 1D with a bond linked to the ANGPTL3 RNAi Drug Substance described in Table 2: as would be understood by the person of ordinary skill in the art, the sodium atoms could alternatively be drawn as Nations.

FIG. 2A to 2D. Chemical structure representation of ANGPTL3 RNAi Drug Substance described in Table 2, shown in a free acid form.

FIG. 3. Schematic diagram of the modified sense and antisense strands of ANGPTL3 RNAi Drug Substance described in Table 2 (referred to herein as ADS-003: i.e., ANGPTL3 RNAi agent conjugated to a tridentate N-acetyl-galactosamine targeting group at the 5′ terminal end of the sense strand). The following abbreviations are used in FIG. 3: a, c, g, and u are 2′-O-methyl modified nucleotides: Af, Cf, Gf, and Uf are 2′-fluoro (also referred to in the art as 2′-deoxy-2′-fluoro) modified nucleotides: p is a phosphodiester linkage: s is a phosphorothioate linkage: invAb is an inverted abasic residue or subunit; and (NAG37) s is a tridentate N-acetyl-galactosamine targeting ligand having the following chemical structure:

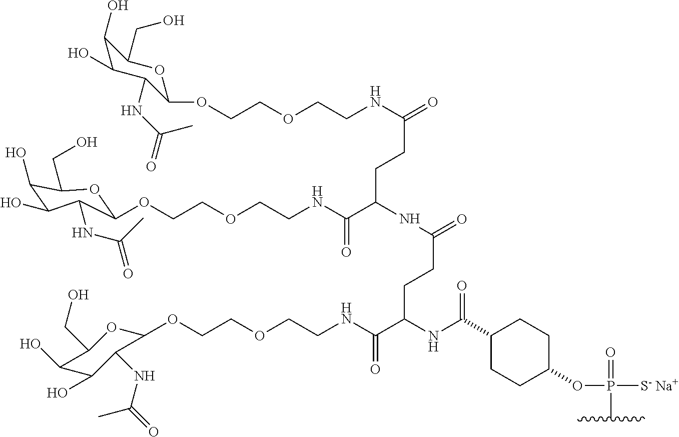

(shown in sodium salt form),

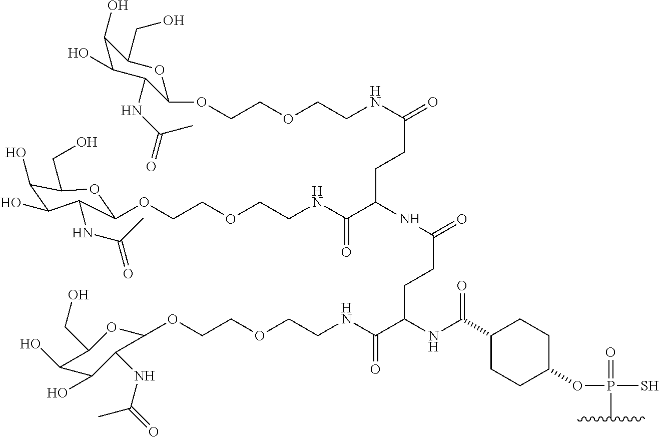

(shown in free acid form).

FIG. 4A. Study schematic for the double-blind treatment period (up through week 36) for the Phase 2b clinical trial described in Example 2.

FIG. 4B. Study schematic for the open-label extension period (beyond week 36) for the Phase 2b clinical trial described in Example 2.

FIG. 5. Dose escalation schedule for the Phase 1 clinical study described in Example 3.

### DETAILED DESCRIPTION

### RNAi Agents

The methods described herein include the administration of a pharmaceutical composition to a human subject, wherein the pharmaceutical composition includes a composition that contains an RNA interference (RNAi) agent (referred to herein and in the art as an RNAi agent or an RNAi trigger) capable of inhibiting expression of an ANGPTL3 gene. In some embodiments, the methods described herein include the administration of a pharmaceutical composition to a human subject, wherein the pharmaceutical composition includes the ANGPTL3 RNAi Drug Substance described in Table 2 (also referred to as ADS-003). The compositions suitable for use in the methods disclosed herein are comprised of an RNAi agent that inhibits expression of an ANGPTL3 gene in a human subject, and a targeting moiety or targeting group. In some embodiments, the RNAi agent includes the nucleotide sequences provided in Table 1A and 1B, and the sense strand of the RNAi agent is further linked or conjugated to a targeting group comprising three N-acetyl-galactosamine targeting moieties (see, e.g., Table B). An RNAi agent that inhibits expression of an ANGPTL3 gene in a human subject is referred to as an “ANGPTL3 RNAi agent.”

In general, ANGPTL3 RNAi agents comprise a sense strand (also referred to as a passenger strand) and an antisense strand (also referred to as a guide strand) that are annealed to form a duplex. The ANGPTL3 RNAi agents disclosed herein include an RNA or RNA-like (e.g., chemically modified RNA) oligonucleotide molecule capable of degrading or inhibiting translation of messenger RNA (mRNA) transcripts of ANGPTL3 mRNA in a sequence specific manner. The ANGPTL3 RNAi agents disclosed herein may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that the ANGPTL3 RNAi agents, as that term is used herein, operate primarily through the RNA interference mechanism, the disclosed RNAi agents are not bound by or limited to any particular pathway or mechanism of action. RNAi agents in general are comprised of a sense strand that is 15 to 49 nucleotides in length and an antisense strand that is 18 to 49 nucleotides in length, and include, but are not limited to: small or short interfering RNAs (siRNAs), double-strand RNAs (dsRNA), micro RNAs (miRNAs), short hairpin RNAs (shRNA), and dicer substrates.

The length of an ANGPTL3 RNAi agent sense strand is typically 15 to 49 nucleotides in length, and the length of an ANGPTL3 RNAi agent antisense strand is typically 17 to 49 nucleotides in length. In some embodiments, the sense and antisense strands are independently 18 to 26 nucleotides in length. In some embodiments, the sense and antisense strands are independently 21 to 26 nucleotides in length. In some embodiments, the sense and antisense strands are independently 21 to 24 nucleotides in length. In some embodiments, the sense strands is independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. In some embodiments, the antisense strand is independently 17, 18, 19, 20, 21, 22, 23, 24, 25, 26, 27, 28, 29, or 30 nucleotides in length. In some embodiments, the sense strand and the antisense strand are both 21 nucleotides in length. The sense and antisense strands can be either the same length or different lengths. The sense and antisense strands can also form overhanging nucleotides on one or both ends of the ANGPTL3 RNAi agent.

ANGPTL3 RNAi agents inhibit, silence, or knockdown ANGPTL3 gene expression. As used herein, the terms “silence.” “reduce,” “inhibit,” “down-regulate,” or “knockdown,” when referring to expression of ANGPTL3, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agent as compared to a second cell, group of cells, tissue, organ, or subject that has not or have not been so treated. In some instances, the reduction in gene expression is measured by comparing the baseline levels of ANGPTL3 mRNA or ANGPTL3 protein in a human subject prior to administration of a composition that comprises an ANGPTL3 RNAi agent, with the ANGPTL3 mRNA or ANGPTL3 protein levels after administration of the therapeutic.

ANGPTL3 gene inhibition, silencing, or knockdown may be measured by any appropriate assay or method known in the art. The non-limiting Examples set forth herein, as well as the examples set forth in International Patent Application Publication No. WO 2019/055633 (Patent Application No. PCT/US2018/050848), which is incorporated by reference herein in its entirety, provide certain examples of appropriate methods to measure ANGPTL3 gene expression inhibition.

ANGPTL3 RNAi agents suitable for use in the methods disclosed herein can be covalently linked or conjugated to a targeting group that includes one or more N-acetyl-galactosamine moieties. In some embodiments, ANGPTL3 RNAi agents suitable for use in the methods disclosed herein are covalently linked or conjugated to a targeting group that includes one or more N-acetyl-galactosamine moieties thereby forming the ANGPTL3 RNAi Drug Substance described in Table 2. In some embodiments, the methods described herein include the administration of the ANGPTL3 RNAi Drug Substance described in Table 2. The ANGPTL3 RNAi Drug Substance described in Table 2 includes the ANGPTL3 RNAi agent shown in Table 1A (antisense strand) and Table 1B (sense strand). The N-acetyl-galactosamine moieties facilitate the targeting of the ANGPTL3 RNAi agent to the asialoglycoprotein receptors (ASGPr) readily present on the surface of hepatocytes, which leads to internalization of the ANGPTL3 RNAi agent by endocytosis or other means.

The ANGPTL3 RNAi agents that can be suitable for use in the methods disclosed herein include an antisense strand that has a region of complementarity to at least a portion of an ANGPTL3 mRNA. ANGPTL3 RNAi agents and ANGPTL3 RNAi Drug Substances suitable for use in the disclosed methods are described in International Patent Application Publication No. WO 2019/055633 (Patent Application No. PCT/US2018/050848), which as previously noted is incorporated by reference herein in its entirety.

The ANGPTL3 mRNA gene transcript for normal humans (referred to as Homo sapiens angiopoietin like 3 (ANGPTL3), mRNA, GenBank NM_014495.4, 2926 bases) as reported in NCBI's GenBank is as follows:

(SEQ ID NO: 1)

| | |
|---|---|
| 1 | agaagaaaac agttccacgt tgcttgaaat tgaaaatcaa |
| | gataaaaatg ttcacaatta |
| 61 | agctccttct ttttattgtt cctctagtta tttcctccag |
| | aattgatcaa gacaattcat |
| 121 | catttgattc tctatctcca gagccaaaat caagatttgc |
| | tatgttagac gatgtaaaaa |
| 181 | ttttagccaa tggcctcctt cagttgggac atggtcttaa |
| | agactttgtc cataagacga |
| 241 | agggccaaat taatgacata tttcaaaaac tcaacatatt |
| | tgatcagtct ttttatgatc |
| 301 | tatcgctgca aaccagtgaa atcaaagaag aagaaaagga |
| | actgagaaga actacatata |
| 361 | aactacaagt caaaaatgaa gaggtaaaga atatgtcact |
| | tgaactcaac tcaaaacttg |
| 421 | aaagcctcct agaagaaaaa attctacttc aacaaaaagt |
| | gaaatattta gaagagcaac |
| 481 | taactaactt aattcaaaat caacctgaaa ctccagaaca |
| | cccagaagta acttcactta |
| 541 | aaacttttgt agaaaaacaa gataatagca tcaaagacct |
| | tctccagacc gtggaagacc |
| 601 | aatataaaca attaaaccaa cagcatagtc aaataaaaga |
| | aatagaaaat cagctcagaa |
| 661 | ggactagtat tcaagaaccc acagaaattt ctctatcttc |
| | caagccaaga gcaccaagaa |
| 721 | ctactccctt tcttcagttg aatgaaataa gaaatgtaaa |
| | acatgatggc attcctgctg |
| 781 | aatgtaccac catttataac agaggtgaac atacaagtgg |
| | catgtatgcc atcagaccca |
| 841 | gcaactctca agtttttcat gtctactgtg atgttatatc |
| | aggtagtcca tggacattaa |
| 901 | ttcaacatcg aatagatgga tcacaaaact tcaatgaaac |
| | gtgggagaac tacaaatatg |
| 961 | gttttgggag gcttgatgga gaattttggt tgggcctaga |
| | gaagatatac tccatagtga |
| 1021 | agcaatctaa ttatgtttta cgaattgagt tggaagactg |
| | gaaagacaac aaacattata |
| 1081 | ttgaatattc tttttacttg ggaaatcacg aaaccaacta |
| | tacgctacat ctagttgcga |
| 1141 | ttactggcaa tgtccccaat gcaatcccgg aaaacaaaga |
| | tttggtgttt tctacttggg |
| 1201 | atcacaaagc aaaaggacac ttcaactgtc cagagggtta |
| | ttcaggaggc tggtggtggc |
| 1261 | atgatgagtg tggagaaaac aacctaaatg gtaaatataa |
| | caaaccaaga gcaaaatcta |
| 1321 | agccagagag gagaagagga ttatcttgga agtctcaaaa |
| | tggaaggtta tactctataa |
| 1381 | aatcaaccaa aatgttgatc catccaacag attcagaaag |
| | ctttgaatga actgaggcaa |
| 1441 | atttaaaagg caataattta aacattaacc tcattccaag |
| | ttaatgtggt ctaataatct |
| 1501 | ggtattaaat ccttaagaga aagcttgaga aatagatttt |
| | ttttatctta aagtcactgt |
| 1561 | ctatttaaga ttaaacatac aatcacataa ccttaaagaa |
| | taccgtttac atttctcaat |
| 1621 | caaaattctt ataatactat ttgttttaaa ttttgtgatg |
| | tgggaatcaa ttttagatgg |
| 1681 | tcacaatcta gattataatc aataggtgaa cttattaaat |
| | aacttttcta aataaaaaat |
| 1741 | ttagagactt ttattttaaa aggcatcata tgagctaata |
| | tcacaacttt cccagtttaa |
| 1801 | aaaactagta ctcttgttaa aactctaaac ttgactaaat |
| | acagaggact ggtaattgta |
| 1861 | cagttcttaa atgttgtagt attaatttca aaactaaaaa |
| | tcgtcagcac agagtatgtg |
| 1921 | taaaaatctg taatacaaat ttttaaactg atgcttcatt |
| | ttgctacaaa ataatttgga |
| 1981 | gtaaatgttt gatatgattt atttatgaaa cctaatgaag |
| | cagaattaaa tactgtatta |
| 2041 | aaataagttc gctgtcttta aacaaatgga gatgactact |
| | aagtcacatt gactttaaca |
| 2101 | tgaggtatca ctatacctta tttgttaaaa tatatactgt |
| | atacatttta tatattttaa |
| 2161 | cacttaatac tatgaaaaca aataattgta aaggaatctt |
| | gtcagattac agtaagaatg |
| 2221 | aacatatttg tggcatcgag ttaaagttta tatttcccct |
| | aaatatgctg tgattctaat |
| 2281 | acattcgtgt aggttttcaa gtagaaataa acctcgtaac |
| | aagttactga acgtttaaac |
| 2341 | agcctgacaa gcatgtatat atgtttaaaa ttcaataaac |
| | aaagacccag tccctaaatt |
| 2401 | atagaaattt aaattattct tgcatgttta tcgacatcac |
| | aacagatccc taaatcccta |
| 2461 | aatccctaaa gattagatac aaatttttta ccacagtatc |
| | acttgtcaga atttattttt |
| 2521 | aaatatgatt ttttaaaact gccagtaaga aattttaaat |
| | taaacccatt tgttaaagga |
| 2581 | tatagtgccc aagttatatg gtgacctacc tttgtcaata |
| | cttagcatta tgtatttcaa |
| 2641 | attatccaat atacatgtca tatatatttt tatatgtcac |
| | atatataaaa gatatgtatg |
| 2701 | atctatgtga atcctaagta aatattttgt tccagaaaag |
| | tacaaaataa taaaggtaaa |
| 2761 | aataatctat aattttcagg accacagact aagctgtcga |
| | aattaacgct gattttttta |
| 2821 | gggccagaat accaaaatgg ctcctctctt cccccaaaat |
| | tggacaattt caaatgcaaa |
| 2881 | attcatt atttaatata tgagttgctt cctctatttg |
| | gtttcc. |

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, the term “complementary,” when used to describe a first nucleotide sequence (e.g., RNAi agent antisense strand) in relation to a second nucleotide sequence (e.g., RNAi agent sense strand or targeted mRNA sequence), means the ability of an oligonucleotide that includes the first nucleotide sequence to hybridize (form base pair hydrogen bonds under mammalian physiological conditions (or otherwise suitable conditions) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that all (100%) of the bases in a contiguous sequence of a first oligonucleotide will hybridize with the same number of nucleotides in a contiguous sequence of a second oligonucleotide. The contiguous sequence may comprise all or a part of a first or second nucleotide sequence.

As used herein. “partially complementary” means that in a hybridized pair of nucleotide sequences, at least 70%, but not all, of the bases in a contiguous sequence of a first oligonucleotide will hybridize with the same number of bases in a contiguous sequence of a second polynucleotide. The contiguous sequence may comprise all or a part of a first or second nucleotide sequence.

As used herein, “substantially complementary” means that in a hybridized pair of nucleotide sequences, at least 85%, but not all, of the bases in a contiguous sequence of a first oligonucleotide will hybridize with the same number of bases in a contiguous sequence of a second polynucleotide. The contiguous sequence may comprise all or a part of a first or second nucleotide sequence.

The terms “complementary.” “fully complementary,” “partially complementary,” and “substantially complementary” herein are used with respect to the nucleotide matching between the sense strand and the antisense strand of an RNAi agent, or between the antisense strand of an RNAi agent and a sequence of an ANGPTL3 mRNA.

As used herein, the term “substantially identical” or “substantially identity” as applied to nucleic acid sequence means that a nucleic acid sequence comprises a sequence that has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

### Modified Nucleotides and Modified Internucleoside Linkages

The ANGPTL3 RNAi agents disclosed herein can be comprised of modified nucleotides, which can preserve activity of the RNAi agent while at the same time increasing the serum stability, as well as minimize the possibility of activating interferon activity in humans. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include any known modified nucleotides known in the art, including but not limited to, deoxyribonucleotides, nucleotide mimics, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-arabino nucleotides, 5′-Me, 2′-fluoro nucleotides, morpholine nucleotides, vinyl phosphonate-containing nucleotides, and cyclopropyl phosphonate-containing nucleotides. In some embodiments, the modified nucleotides of an ANGPTL3 RNAi agent are 2′-modified nucleotides (i.e. a nucleotide with a group other than a hydroxyl group at the 2′ position of the five-membered sugar ring). 2′-modified nucleotides include, but are not limited to, 2′-O-methyl nucleotides, 2′-deoxy-2′-fluoro nucleotides (commonly referred to simply as 2′-fluoro nucleotides), 2′-deoxy nucleotides, 2′-methoxyethyl (2′-O-2-methoxyethyl) nucleotides, 2′-amino nucleotides, and 2′-alkyl nucleotides. Additional 2′-modified nucleotides are known in the art. It is not necessary for all nucleotides in a given RNAi agent to be uniformly modified. Additionally, more than one modification can be incorporated in a single ANGPTL3 RNAi agent or even in a single nucleotide thereof. The ANGPTL3 RNAi agent sense strands and antisense strands can be synthesized and/or modified by methods known in the art. Modification at one nucleotide is independent of modification at another nucleotide.

In some embodiments, the nucleobase may be modified. As is commonly used in the art, natural nucleobases include the primary purine bases adenine and guanine, and the primary pyrimidine bases cytosine, thymine, and uracil. A nucleobase may be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

Modified nucleobases include, for example, 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an ANGPTL3 RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0), 1, or 2) nucleotides in the sense strand being ribonucleotides. As used herein, an antisense sense strand wherein substantially all of the nucleotides present are modified nucleotides is an antisense strand having two or fewer (i.e., 0), 1, or 2) nucleotides in the antisense strand being ribonucleotides.

In some embodiments, one or more nucleotides of an ANGPTL3 RNAi agent are linked by non-standard linkages or backbones (i.e., modified internucleoside linkages or modified backbones). Modified internucleoside linkages or backbones include, but are not limited to, phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an ANGPTL3 RNAi agent can contain 1, 2, 3, 4, 5, or 6 phosphorothioate linkages, an antisense strand of an ANGPTL3 RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an ANGPTL3 RNAi agent can contain 1, 2, 3, or 4 phosphorothioate linkages, an antisense strand of an ANGPTL3 RNAi agent can contain 1, 2, 3, or 4 phosphorothioate linkages, or both the sense strand and the antisense strand independently can contain 1, 2, 3, or 4 phosphorothioate linkages.

In some embodiments, an ANGPTL3 RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, phosphorothioate internucleoside linkages are used to link the terminal nucleotides in the sense strand to capping residues present at the 5′-end, the 3′-end, or both the 5′- and 3′-ends of the nucleotide sequence. In some embodiments, phosphorothioate internucleoside linkages are used to link a targeting group to the sense strand.

In some embodiments, an ANGPTL3 RNAi agent antisense strand contains three or four phosphorothioate internucleoside linkages. In some embodiments, an ANGPTL3 RNAi agent antisense strand contains four phosphorothioate internucleoside linkages. In some embodiments, three phosphorothioate internucleoside linkages are between the nucleotides at positions 1-4 from the 5′ end of the antisense strand and between the nucleotides at positions 19-21, 20-22, 21-23, 22-24, 23-25, or 24-26 from the 5′ end. In some embodiments, an ANGPTL3 RNAi agent contains at least two phosphorothioate internucleoside linkages in the sense strand and three or four phosphorothioate internucleoside linkages in the antisense strand.

In some embodiments, an ANGPTL3 RNAi agent contains one or more modified nucleotides and one or more modified internucleoside linkages. In some embodiments, a 2′-modified nucleoside is combined with modified internucleoside linkage.

### Capping Residues or Moieties

In some embodiments, the sense strand may include one or more capping residues or moieties, sometimes referred to in the art as a “cap,” a “terminal cap,” or a “capping residue,” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table A). (See, e.g., F. Czauderna, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

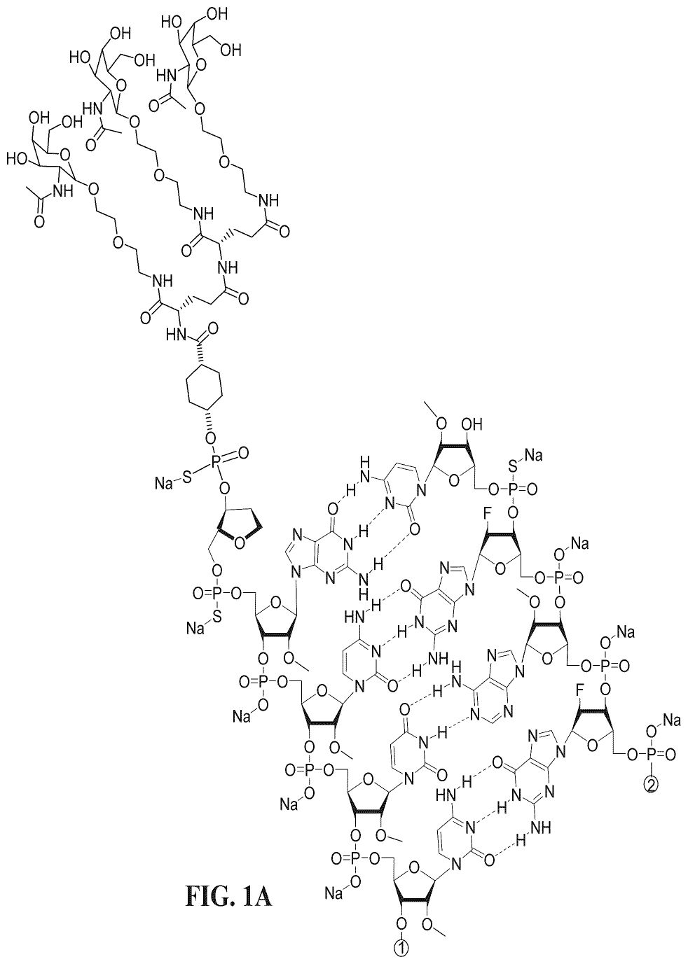

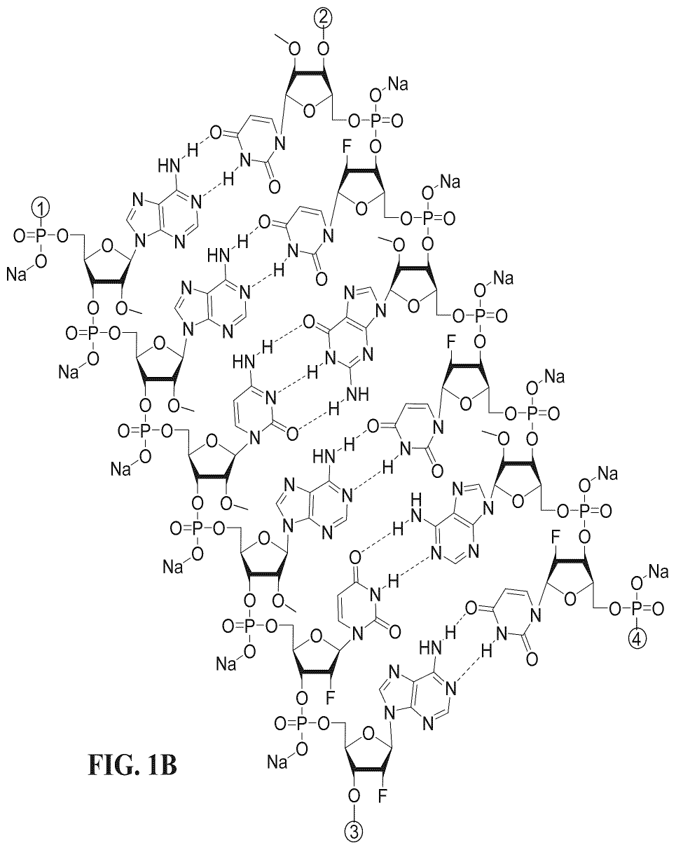

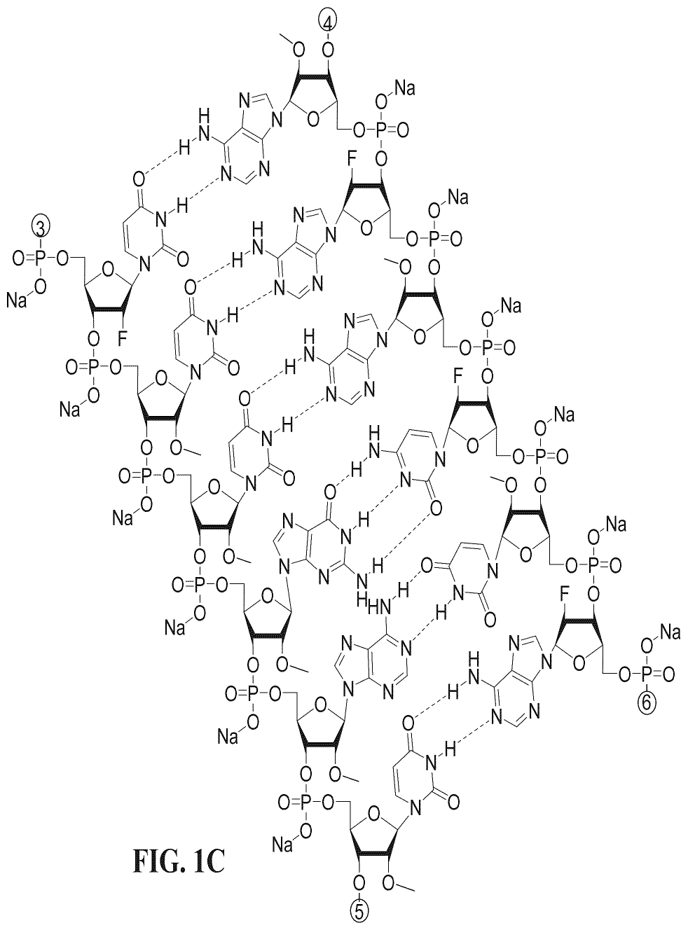

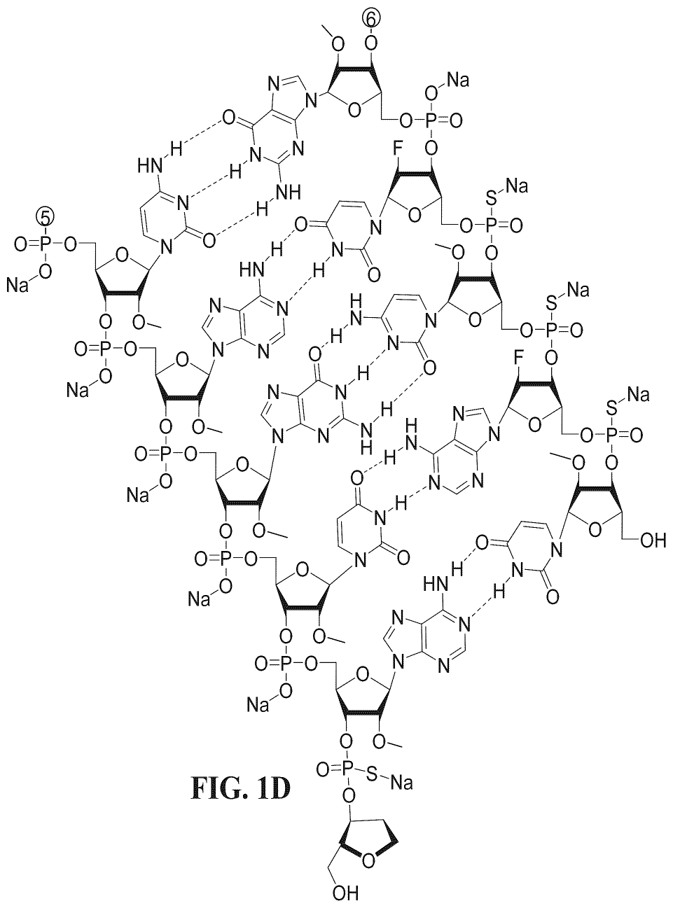

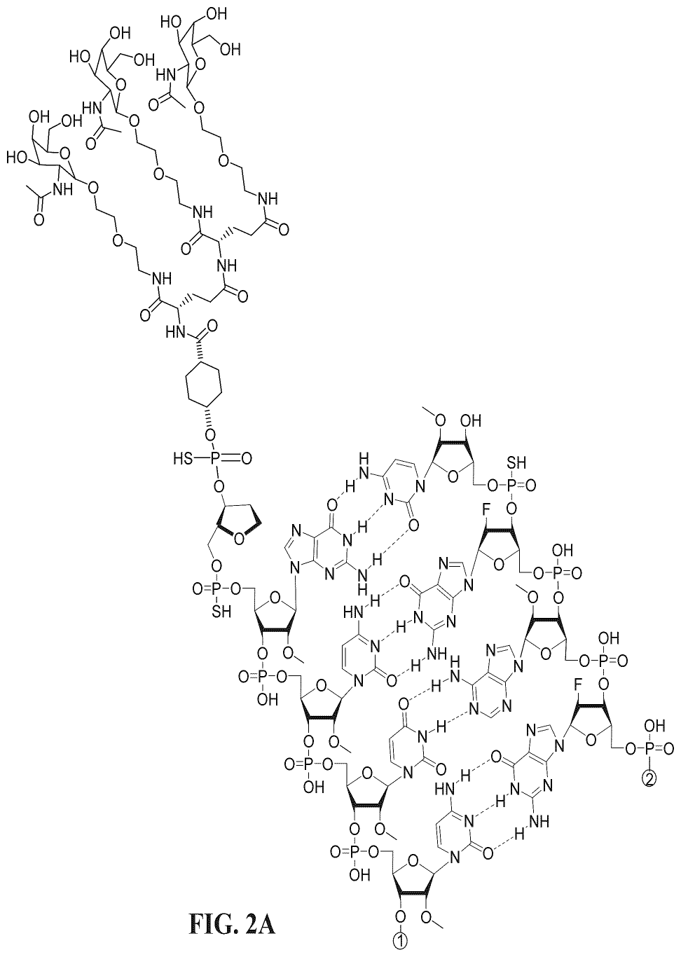

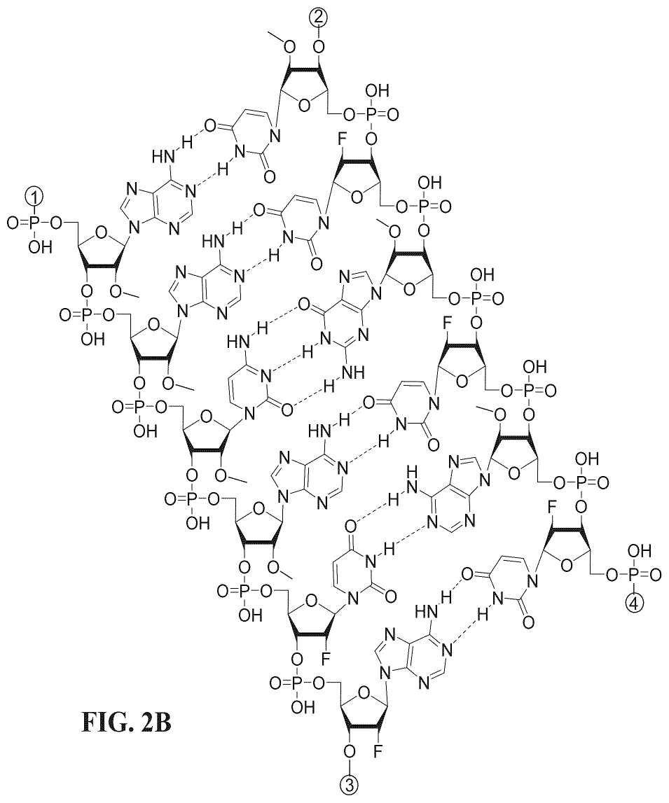

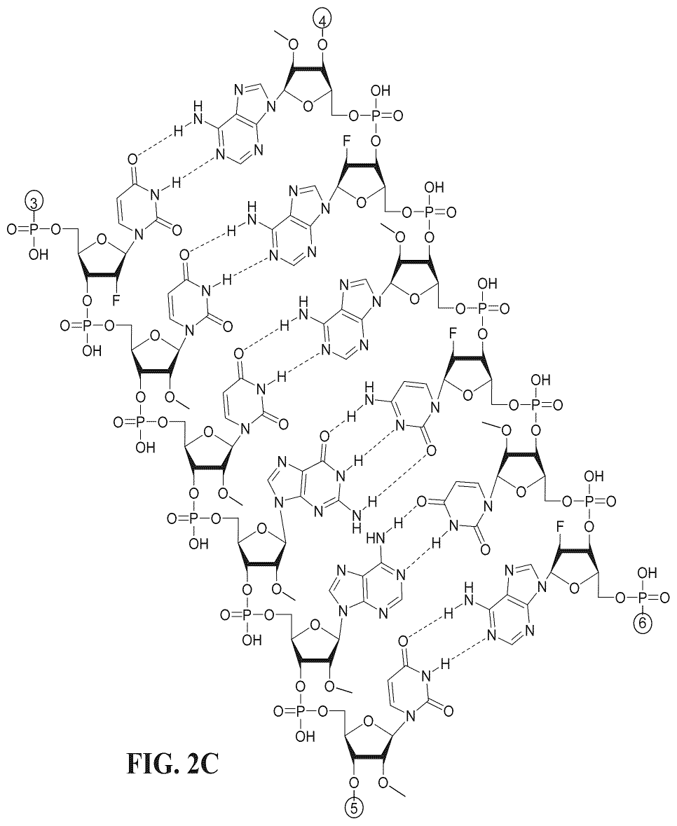

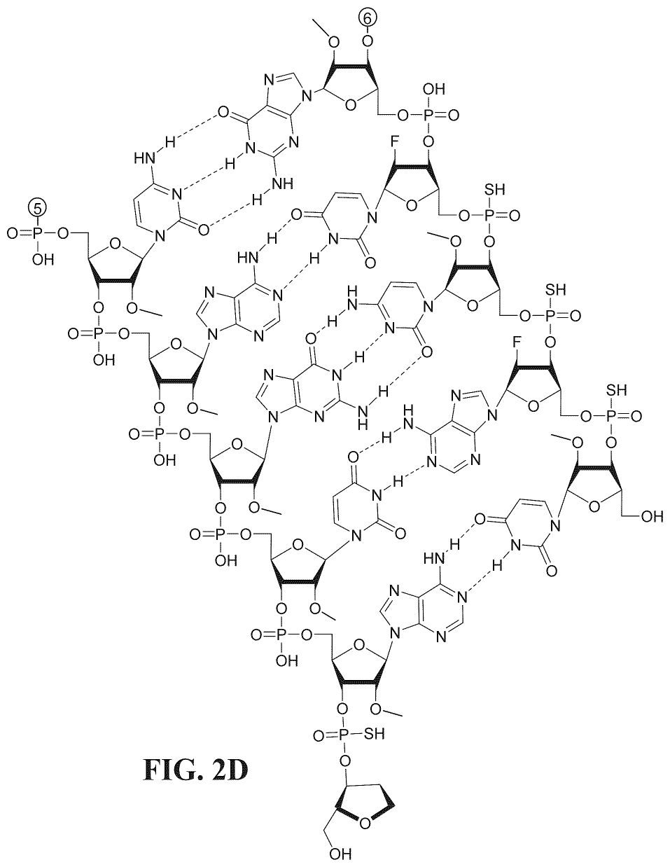

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb) s)), or other internucleoside linkages. The chemical structures for inverted abasic deoxyribose residues are shown in Table A below, as well as in the chemical structures shown in FIGS. 1A to ID and FIGS. 2A to 2D.

TABLE A
Inverted Abasic (Deoxyribose) Chemical Structures

When positioned internally on oligonucleotide:
linkage towards 5' end of
oligonucleotide

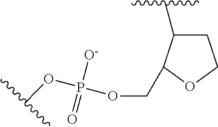 [SMILES: *OP(=O)([O-])OCC1OCCC1*]

linkage towards 3' end of
oligonucleotide
(invAb)
When positioned internally on oligonucleotide:
linkage towards 5' end of
oligonucleotide

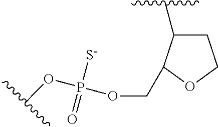 [SMILES: *OP(=O)([S-])OCC1OCCC1*]

linkage towards 3' end of
oligonucleotide
(invAb)s
When positioned at the 3' terminal end of oligonucleotide:
linkage towards 5' end of
oligonucleotide

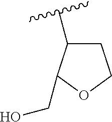 [SMILES: *C1CCOC1CO]

(invAb)

### Targeting Moieties and Groups

An ANGPTL3 RNAi agent can be conjugated to one or more non-nucleotide groups including, but not limited to, a targeting moiety or a targeting group. A targeting moiety or targeting group can enhance targeting or delivery of the RNAi agent. Examples of targeting moieties and targeting groups are known in the art. Specific examples of the (NAG37) s targeting group used in the ANGPTL3 RNAi Drug Substance described in Table 2 herein, which includes three N-acetyl-galactosamine targeting moieties, is provided in Table B. The targeting moiety or targeting group can be covalently linked to the 3′ and/or 5′ end of either the sense strand and/or the antisense strand. In some embodiments, an ANGPTL3 RNAi agent contains a targeting group linked to the 3′ and/or 5′ end of the sense strand. In some embodiments, a targeting group is linked to the 5′ end of an ANGPTL3 RNAi agent sense strand. In some embodiments, the targeting group comprises, consists essential of, or consists of the structure (NAG37)s, and is linked to the 5′ end of an ANGPTL3 RNAi agent sense strand. A targeting group can be linked directly or indirectly to the RNAi agent via a linker/linking group. In some embodiments, a targeting group is linked to the RNAi agent via a labile, cleavable, or reversible bond or linker. In some embodiments, a targeting group is linked to an inverted abasic residue at the 5′ end of the sense strand.

Targeting groups or targeting moieties can enhance the pharmacokinetic or biodistribution properties of a conjugate or RNAi agent to which they are attached to improve cell-specific distribution and cell-specific uptake of the conjugate or RNAi agent. In some embodiments, a targeting group enhances endocytosis of the RNAi agent. A targeting group can be monovalent, divalent, trivalent, tetravalent, or have higher valency for the target to which it is directed. Representative targeting groups include, without limitation, compounds with affinity to cell surface molecules, cell receptor ligands, haptens, antibodies, monoclonal antibodies, antibody fragments, and antibody mimics with affinity to cell surface molecules.

In some embodiments, a targeting group comprises an asialoglycoprotein receptor ligand. In some embodiments, an asialoglycoprotein receptor ligand includes or consists of one or more galactose derivatives. As used herein, the term galactose derivative includes both galactose and derivatives of galactose having affinity for the asialoglycoprotein receptor that is equal to or greater than that of galactose. Galactose derivatives include, but are not limited to: galactose, galactosamine. N-formylgalactosamine, N-acetyl-galactosamine, N-propionyl-galactosamine, N-n-butanoyl-galactosamine, and N-iso-butanoyl-galactosamine (see for example: S. T. Iobst and K. Drickamer. J. B. C., 1996, 271, 6686). Galactose derivatives, and clusters of galactose derivatives, that are useful for in vivo targeting of oligonucleotides and other molecules to the liver are known in the art (see, for example. Baenziger and Fiete. 1980). Cell, 22, 611-620; Connolly et al., 1982, J. Biol. Chem., 257, 939-945).

Galactose derivatives have been used to target molecules to hepatocytes in vivo through their binding to the asialoglycoprotein receptor expressed on the surface of hepatocytes. Binding of asialoglycoprotein receptor ligands to the asialoglycoprotein receptor(s) facilitates cell-specific targeting to hepatocytes and endocytosis of the molecule into hepatocytes. Asialoglycoprotein receptor ligands can be monomeric (e.g., having a single galactose derivative) or multimeric (e.g., having multiple galactose derivatives). The galactose derivative or galactose derivative “cluster” can be attached to the 3′ or 5′ end of the sense or antisense strand of the RNAi agent using methods known in the art.

In some embodiments, a targeting group comprises a galactose derivative cluster. As used herein, a galactose derivative cluster comprises a molecule having two to four terminal galactose derivatives. A terminal galactose derivative is attached to a molecule through its C-1 carbon. In some embodiments, the galactose derivative cluster is a galactose derivative trimer (also referred to as tri-antennary galactose derivative or tri-valent galactose derivative). In some embodiments, the galactose derivative cluster comprises N-acetyl-galactosamines. In some embodiments, the galactose derivative cluster comprises three N-acetyl-galactosamines. In some embodiments, the galactose derivative cluster is a galactose derivative tetramer (also referred to as tetra-antennary galactose derivative or tetra-valent galactose derivative). In some embodiments, the galactose derivative cluster comprises four N-acetyl-galactosamines.

As used herein, a galactose derivative trimer contains three galactose derivatives, each linked to a central branch point. As used herein, a galactose derivative tetramer contains four galactose derivatives, each linked to a central branch point. The galactose derivatives can be attached to the central branch point through the C-1 carbons of the saccharides. In some embodiments, the galactose derivatives are linked to the branch point via linkers or spacers. In some embodiments, the linker or spacer is a flexible hydrophilic spacer, such as a PEG group (see, for example. U.S. Pat. No. 5,885,968: Biessen et al. J. Med. Chem. 1995 Vol. 39 p. 1538-1546). The branch point can be any small molecule which permits attachment of three galactose derivatives and further permits attachment of the branch point to the RNAi agent. An example of branch point group is a di-lysine or di-glutamate. Attachment of the branch point to the RNAi agent can occur through a linker or spacer. In some embodiments, the linker or spacer comprises a flexible hydrophilic spacer, such as, but not limited to, a PEG spacer. In some embodiments, the linker comprises a rigid linker, such as a cyclic group. In some embodiments, a galactose derivative comprises or consists of N-acetyl-galactosamine. In some embodiments, the galactose derivative cluster is comprised of a galactose derivative tetramer, which can be, for example, an N-acetyl-galactosamine tetramer.

The preparation of targeting groups, such as galactose derivative clusters that include N-acetyl-galactosamine, is described in, for example. International Patent Application Publication No. WO 2018/044350 (Patent Application No. PCT/US2017/021147) and International Patent Application Publication No. WO 2017/156012 (Patent Application No. PCT/US2017/021175), the contents of both of which are incorporated by reference herein in their entirety.

For example, the targeting ligand conjugated to the ANGPTL3 RNAi agent described in Tables 1A and 1B has the chemical structure of (NAG37) s, as shown in the following Table B.

TABLE B
Chemical Structure of (NAG37)s.

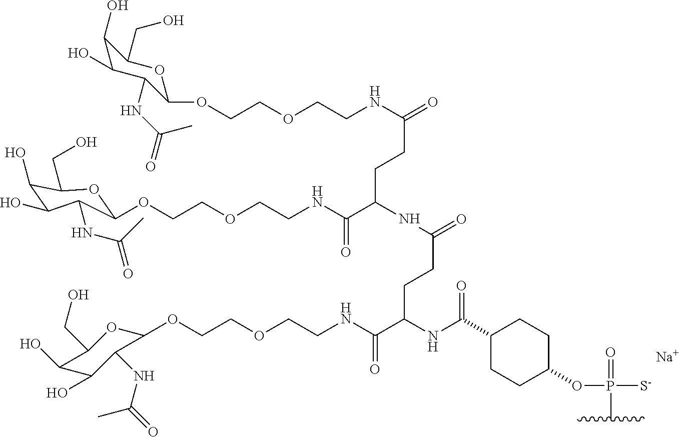

((NAG37)s shown in sodium salt form)

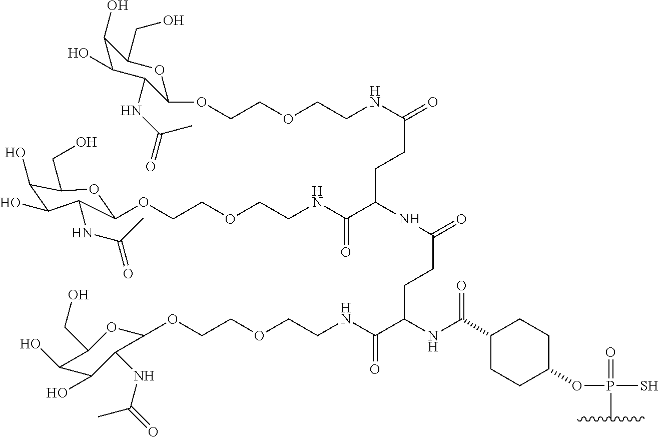

((NAG37)s shown in free acid form)

### ANGPTL3 RNAi Agents and ANGPTL3 RNAi Drug Substance (ADS-003)

In some embodiments, the ANGPTL3 RNAi agent used in the methods disclosed herein have the nucleotide sequences of the ANGPTL3 RNAi Drug Substance (ADS-003) shown in Table 2. The nucleotide sequences of the ANGPTL3 RNAi agent found in ANGPTL3 RNAi Drug Substance include an antisense strand nucleotide sequence as set forth in the following Table 1A, and a sense strand nucleotide sequence as set forth in the following Table 1B.

TABLE 1A
ANGPTL3 RNAi Agent Antisense Strand Sequence

| SEQ ID | Antisense Sequence (Modified) | SEQ | Underlying Base Sequence |
|---|---|---|---|
| NO. | (5′ → 3′) | ID NO. | (5′ → 3′) |
| 2 | usAfscsUfgAfuCfaAfaUfaUfgUfuGfaGfsc | 3 | UACUGAUCAAAUAUGUUGAGC |

TABLE 1B
ANGPTL3 RNAi Agent Sense Strand Nucleotide Sequence (shown as modified
version without inverted abasic residues or NAG targeting group present in
ANGPTL3 RNAi Drug Substance)

| SEQ | Sense Sequence (Modified) | SEQ ID | Underlying Base Sequence |
|---|---|---|---|
| ID NO. | (5′ → 3′) | NO. | (5′ → 3′) |
| 4 | gcucaacaUfAfUfuugaucagua | 5 | GCUCAACAUAUUUGAUCAGUA |

As used in Tables 1A, 1B, and 2 herein, the following notations are used to indicate modified nucleotides, targeting groups, and linking groups: A, C, G, and U represent adenosine, cytidine, guanosine, and uridine, respectively: a, c, g, and u represent 2′-O-methyl adenosine, cytidine, guanosine, and uridine, respectively: Af, Cf, Gf, and Uf represent 2′-fluoro adenosine, cytidine, guanosine, and uridine, respectively: s represents a phosphorothioate linkage: (invAb) represents an inverted abasic deoxyribose residue (see Table A); and (NAG37) s represents the structure shown in Table B, above.

As the person of ordinary skill in the art would readily understand, unless otherwise indicated by the sequence (such as, for example, by a phosphorothioate linkage “s”), when present in a strand, the monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides (see, e.g., FIGS. 1-3 showing all linkages). Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand. Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the ANGPTL3 RNAi agents and compositions that include ANGPTL3 RNAi agents disclosed herein.

Each sense strand and/or antisense strand can have any targeting groups or linking groups listed above, as well as other targeting or linking groups, conjugated to the 5′ and/or 3′ end of the sequence.

The ANGPTL3 RNAi agent antisense strand sequence is designed to target mRNA transcripts from an ANGPTL3 gene in a human subject, thereby silencing translation of ANGPTL3 protein using an RNA interference mechanism for human subjects with ANGPTL3.

In some embodiments, the methods disclosed herein use the ANGPTL3 RNAi Drug Substance set forth in the following Table 2:

TABLE 2
ANGPTL3 RNAi Drug Substance (ADS-003)
Sense and Antisense Strands (The sense and antisense strands are annealed to form a duplex):

| Sense Strand (Modified Sequence) | (NAG37)s(invAb)sgcucaacaUfAfUfuugaucaguas(invAb) | (SEQ ID NO: 6) |
|---|---|---|
| (5′ → 3′): | | |
| Antisense Strand (Modified Sequence) | usAfscsUfgAfuCfaAfaUfaUfgUfuGfaGfsc | (SEQ ID NO: 2) |
| (5′ → 3′): | | |

TABLE 2.1
Properties of ANGPTL3 RNAi Drug Substance
(ADS-003) Described in Table 2

| | |
|---|---|
| Chemical Formula: | C₄₉₃H₆₀₃F₁₃N₁₆₂Na₄₃O₃₀₉P₄₃S₇ (Na⁺ form) |
| | C₄₉₃H₆₄₆F₁₃N₁₆₂O₃₀₉P₄₃S₇ (H⁺ form) |
| Molecular Weight: | 16509.8 Da (Na⁺ form) |
| | 15587.6 Da (H⁺ form) |
| Physical Appearance: | White to Off-white Powder |

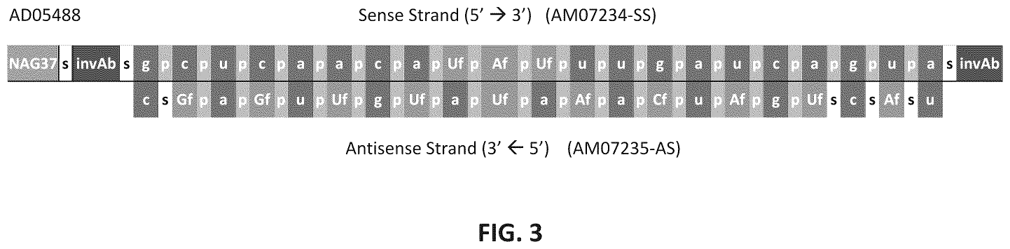

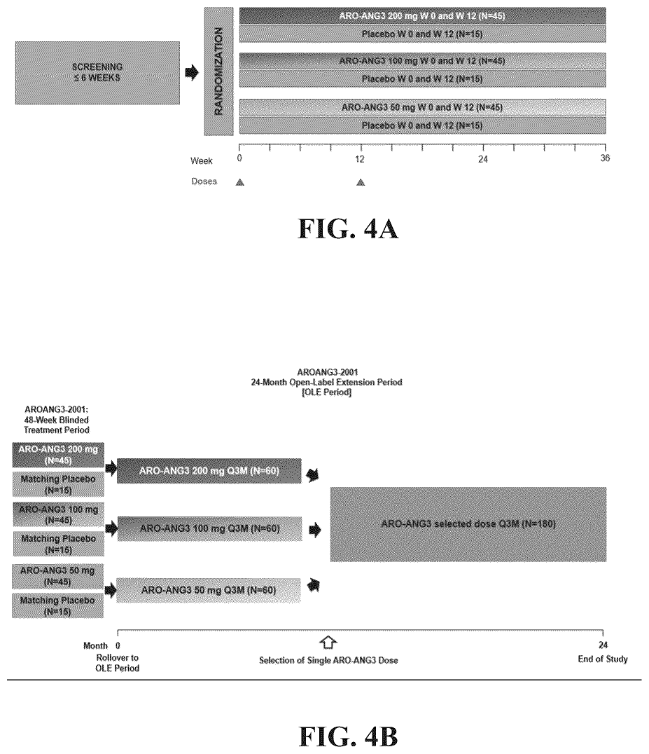

A schematic representation of ANGPTL3 RNAi Drug Substance (ADS-003) is shown in FIG. 3, and full chemical structure representations are shown in FIGS. 1A to 1D (sodium salt form) and FIGS. 2A to 2D free acid form). In some embodiments, the ANGPTL3 RNAi Drug Substance is prepared or provided as a salt, mixed salt, or a free acid. In some embodiments, the form is a sodium salt.

Unless expressly mentioned otherwise herein, the dosage amount (e.g., 50 mg, 100 mg, 200 mg. 300 mg, etc.) of the ANGPTL3 RNAi Drug Substance used in the Examples disclosed herein was calculated based on the free acid form.

### PHARMACEUTICAL COMPOSITIONS AND FORMULATIONS

The ANGPTL3 RNAi agents suitable for use in the methods disclosed herein can be prepared as pharmaceutical compositions or formulations for administration to human subjects. The pharmaceutical compositions can be used to treat a subject having a disease or disorder that would benefit from inhibition of expression of ANGPTL3 mRNA or reduction in the level of ANGPTL3 protein, such as human subjects having an ANGPTL3-related disease or disorder. In some embodiments, the methods include administering an ANGPTL3 RNAi agent that is linked to a targeting group or targeting ligand as described herein, to a subject in need of treatment. In some embodiments, one or more pharmaceutically acceptable excipients (including vehicles, carriers, diluents, and/or delivery polymers) are added to the pharmaceutical compositions that include an ANGPTL3 RNAi agent, thereby forming a pharmaceutical formulation suitable for in vivo delivery to a human subject.

The pharmaceutical compositions that include an ANGPTL3 RNAi agent, when administered to a human subject using the methods disclosed herein, decrease or reduce the level of ANGPTL3 mRNA in the subject.

In some embodiments, the described pharmaceutical compositions including an ANGPTL3 RNAi agent are used for treating or managing clinical presentations in a subject with an ANGPTL3-related disease or disorder. In some embodiments, a therapeutically or prophylactically effective amount of one or more of pharmaceutical compositions is administered to a subject in need of such treatment. In some embodiments, administration of any of the disclosed ANGPTL3 RNAi agents can be used to decrease the number, severity, and/or frequency of symptoms of a disease in a subject.

The described pharmaceutical compositions that include an ANGPTL3 RNAi agent can be used to treat at least one symptom in a subject having a disease or disorder that would benefit from reduction or inhibition in expression of ANGPTL3 protein levels. In some embodiments, the subject is administered a therapeutically effective amount of one or more pharmaceutical compositions including an ANGPTL3 RNAi agent thereby treating the symptom. In other embodiments, the subject is administered a prophylactically effective amount of one or more ANGPTL3 RNAi agents, thereby preventing the at least one symptom.

The ANGPTL3 RNAi agents disclosed herein can be administered via any suitable route in a preparation appropriately tailored to the particular route. Thus, herein described pharmaceutical compositions can be administered by injection, for example, intravenously or subcutaneously. In some embodiments, the herein described pharmaceutical compositions are administered via subcutaneous injection.

As used herein, a pharmaceutical composition or medicament includes a pharmacologically effective amount of at least one ANGPTL3 RNAi agents and one or more pharmaceutically acceptable excipients. Pharmaceutically acceptable excipients (excipients) are substances other than the Active Pharmaceutical Ingredient (API, therapeutic product, e.g., ANGPTL3 RNAi agent) that are intentionally included in the drug delivery system. Excipients do not exert or are not intended to exert a therapeutic effect at the intended dosage. Excipients can act to a) aid in processing of the drug delivery system during manufacture, b) protect, support, or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients may include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble). For subcutaneous or intravenous administration, suitable carriers may include physiological saline, bacteriostatic water, Cremophor® ELTM (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle, which contains a basic dispersion medium and the required other ingredients from those enumerated above.

In some embodiments, a pharmaceutical composition suitable for use in the methods disclosed herein includes the components identified in the Formulated ANGPTL3 RNAi Drug Substance provided in Table 3, below.

The ANGPTL3 RNAi agents can be formulated in compositions in dosage unit form for ease of administration and uniformity of dosage. Dosage unit form refers to physically discrete units suited as unitary dosages for the subject to be treated: each unit containing a predetermined quantity of active compound calculated to produce the desired therapeutic effect in association with the required pharmaceutical carrier. In some embodiments, the dosage unit is between about 50 mg and about 400 mg of ANGPTL3 RNAi Drug Substance. In some embodiments, the dosage unit is between about 100 mg and about 300 mg of ANGPTL3 RNAi Drug Substance. In some embodiments, the dosage unit is between about 200 mg and about 300 mg of ANGPTL3 RNAi Drug Substance. In some embodiments, the dosage unit is at least 50 mg of ANGPTL3 RNAi Drug Substance. In some embodiments, the dosage unit is about 200 mg of ANGPTL3 RNAi Drug Substance. In some embodiments, the dosage unit is about 300 mg of ANGPTL3 RNAi Drug Substance. In some embodiments, the dosage unit is about 100 mg of ANGPTL3 RNAi Drug Substance. In some embodiments, the dosage unit is from about 50, 75, 100, 125, 150, 175, 200, 225, 250, 275, 300, 325, 350, or 375 mg to about 75, 100, 125, 150, 175, 200, 225, 250, 275, 300, 325, 350, 375, or 400 mg of ANGPTL3 RNAi Drug Substance.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.).

As used herein, “pharmacologically effective amount,” “therapeutically effective amount,” or simply “effective amount” refers to that amount of an RNAi agent to produce a pharmacological, therapeutic or preventive result.

The described pharmaceutically acceptable formulations can be packaged into kits, containers, packs, or dispensers. The pharmaceutical compositions described herein can be packaged in pre-filled syringes or vials.

### Formulated ANGPTL3 RNAi Drug Substance

In some embodiments, the ANGPTL3 RNAi Drug Substance as provided in Table 2 (ADS-003) is formulated with one or more pharmaceutically acceptable excipients to form a pharmaceutical composition suitable for administration to a human subject. In some embodiments, the ANGPTL3 RNAi Drug Substance described in Table 2 is formulated at 200 mg/mL in an aqueous sodium phosphate buffer (0.5 mM sodium phosphate monobasic, 0.5 mM sodium phosphate dibasic), forming the Formulated ANGPTL3 RNAi Drug Substance (ADS-003-1) shown in Table 3:

TABLE 3
Composition of Formulated ANGPTL3
RNAi Drug Substance, per 1.0 mL

| | | Quality/ | | |
|---|---|---|---|---|
| Component | Function | Grade | Concentration | |
| ADS-003 | Active ingredient | In-house | 200 | mg |
| Sodium phosphate | Suspending agent | USP, Ph. | 0.061 | mg |
| monobasic, monohydrate | | Eur | | |
| Sodium phosphate | Suspending agent | USP, Ph. | 0.062 | mg |
| dibasic, anhydrous | | Eur | | |
| Water for injection | Vehicle | USP, Ph. | 879.2 | mg |
| (WFI) | | Eur | | |

The Formulated ANGPTL3 RNAi Drug Substance according to Table 3 is prepared as a sterile formulation. In some embodiments, the Formulated ANGPTL3 RNAI Drug Substance is packaged in a container, such as a glass vial. In some embodiments, the Formulated ANGPTL3 RNAi Drug Substance is packaged in a glass vial with a fill volume of about 1.2 mL, and a desired volume for administration can be calculated based upon the desired dose level to be administered.

In some embodiments, the Formulated ANGPTL3 RNAi Drug Substance set forth in Table 3 is administered to a human subject using the methods disclosed herein.

Human Subjects with Elevated Triglyceride Levels and/or Over-Expression of ANGPTL3

The methods disclosed herein include treating diseases, disorders, or other conditions or symptoms that can be ameliorated at least in part by a reduction in or the silencing of ANGPTL3 gene expression in a human subject in need thereof, using pharmaceutical compositions that include the ANGPTL3 RNAi Drug Substance described in Table 2. In some embodiments, the methods disclosed herein include treating diseases or conditions associated with elevated TG and/or LDL-C in a human subject in need thereof, using pharmaceutical compositions that include the ANGPTL3 RNAi Drug Substance described in Table 2. In some embodiments, prior to administration the human subject is diagnosed with an ANGPTL3-related disease or disorder. In some embodiments, the human subject has been diagnosed as having cardiovascular disease, coronary artery disease, or atherosclerosis. In some embodiments, the human subject has been diagnosed as having moderate hypertriglyceridemia (TG levels of 150-499 mg/dL according to the 2018 AHA/ACC Guidelines), severe hypertriglyceridemia (TG>500 mg/dL), and/or chylomicronemia, familial chylomicronemia syndrome (FCS) (TG levels often >1000 mg/dL), mixed dyslipidemia, and/or homozygous familial hypercholesterolemia (HoFH). HoFH is a severe, rare genetic disease, with a prevalence of 1 in ˜250,000, in which an individual has two inherited causal familial hypercholesterolemia (FH) variants, and often leads to a more serious form of FH disease in which the individual exhibits extremely high levels of LDL-C(usually above 400 mg/dL) that can cause advanced coronary artery disease in the individual as early as the age of 20. As shown in the Examples herein, the ANGPTL3 RNAi Drug Substance described in Table 2 provides for a substantial TG and LDL-C lowering effect suitable for the treatment of diseases and disorders, while also not resulting in a substantial increase in undesired liver fat or AST or ALT enzyme elevations.

### Dosing and Inhibition of ANGPTL3 Gene Expression

Generally, an effective amount of an ANGPTL3 RNAi agent will be in the range of from about 0.1 to about 10 mg/kg of body weight per dose, e.g., from about 0.25 to about 5 mg/kg of body weight per dose. In some embodiments, an effective amount of an ANGPTL3 RNAi agent will be in the range of from about 0.5 to about 4 mg/kg of body weight per dose. In some embodiments, the effective amount is a fixed dose. In some embodiments, a fixed dose of between 50 mg to 400 mg of ANGPTL3 RNAi Drug Substance is an effective dose. In some embodiments, a fixed dose of between 100 mg to 300 mg of ANGPTL3 RNAi Drug Substance is an effective dose. In some embodiments, a fixed dose of between 200 mg to 300 mg of ANGPTL3 RNAi Drug Substance is an effective dose. In some embodiments, a fixed dose of between 200 mg ANGPTL3 RNAi Drug Substance is an effective dose. In some embodiments, a fixed dose of between 300 mg ANGPTL3 RNAi Drug Substance is an effective dose. The amount administered will likely depend on such variables as the overall age and health status of the patient, the relative biological efficacy of the compound delivered, the formulation of the drug, the presence and types of excipients in the formulation, and the route of administration. In some embodiments, a fixed dose of about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg is an effective dose.

Also, it is to be understood that the initial dosage administered can, in some instances, be increased beyond the above upper level to rapidly achieve the desired blood-level or tissue level, or the initial dosage can, in some instances, be smaller than the optimum. For example, in some embodiments, an initial dose or a first dose of from about 50 mg to about 400 mg of ANGPTL3 RNAi Drug Substance is administered, followed by a second dose of from about 50 to 300 mg of ANGPTL3 RNAi Drug Substance approximately 1 month later, and thereafter additional doses (a concept similar to “maintenance doses”) are administered once every about three months (e.g., once per calendar quarter or once per 12 weeks (q12w)). In some embodiments, an initial dose or a first dose of from about 50 mg to about 400 mg of ANGPTL3 RNAi Drug Substance is administered, followed by a second dose of from about 50 to 300 mg of ANGPTL3 RNAi Drug Substance approximately 1 month later, and thereafter additional doses (a concept similar to “maintenance doses”) are administered once every six months (e.g., twice per calendar year or q6m).

For treatment of disease or for formation of a medicament or composition for treatment of a disease, the pharmaceutical compositions described herein including an ANGPTL3 RNAi agent can be combined with an excipient or with a second therapeutic agent or treatment including, but not limited to: a second or other RNAi agent, a small molecule drug such as a statin, a PCSK9-inhibitor, an antibody, an antibody fragment, peptide and/or aptamer.

In some embodiments, the ANGPTL3 mRNA level of a subject to whom a described ANGPTL3 RNAi agent is administered is reduced in the liver by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%. In some embodiments, the ANGPTL3 mRNA level of a subject to whom a described ANGPTL3 RNAi agent is administered is reduced in the entire subject by at least about 5%, 10%, 15%, 20%, 25%, 30%, 35%, 40%, 45%, 50%, 55%, 60%, 65%, 70%, 75%, 80%, or greater than 80% relative to the subject prior to being administered the ANGPTL3 RNAi agent or to a subject not receiving the ANGPTL3 RNAi agent. The gene expression level and/or mRNA level in the subject is reduced in a cell, group of cells, and/or tissue of the subject.

In some embodiments, the ANGPTL3 protein levels in a subject to whom a described ANGPTL3 RNAi agent has been administered is reduced in the liver by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the ANGPTL3 RNAi agent or to a subject not receiving the ANGPTL3 RNAi agent. In some embodiments, the ANGPTL3 protein levels in a subject to whom a described ANGPTL3 RNAi agent has been administered is reduced in the entire subject by at least about 5%, 10%, 15%, 20%, 25%, 30%, 35%, 40%, 45%, 50%, 55%, 60%, 65%, 70%, 75%, 80%, or greater than 80% relative to the subject prior to being administered the ANGPTL3 RNAi agent or to a subject not receiving the ANGPTL3 RNAi agent. The protein level in the subject can be reduced in a cell, group of cells, tissue, blood, and/or other fluid of the subject.

A reduction in ANGPTL3 gene expression, ANGPTL3 mRNA, or ANGPTL3 protein levels can be assessed and quantified by general methods known in the art. The Examples disclosed in WO 2019/055633 (Patent Application No. PCT/US2018/050848) set forth generally appropriate and known methods for assessing inhibition of ANGPTL3 gene expression and reduction in ANGPTL3 protein levels. The reduction or decrease in ANGPTL3 mRNA level and/or protein level are collectively referred to herein as a reduction or decrease in ANGPTL3 or inhibiting or reducing the expression of ANGPTL3.

In some embodiments, the triglyceride (TG) level in a subject to whom a described ANGPTL3 RNAi agent is administered is reduced by at least about 5%, 10%, 15%, 20%, 25%, 30%, 35%, 40%, 45%, 50%, 55%, 60%, 65%, 70%, or greater than 70% relative to the subject prior to being administered the ANGPTL3 RNAi agent or to a subject not receiving the ANGPTL3 RNAi agent. The triglyceride level in the subject is reduced in the blood/serum of the subject.

In some embodiments, the low density lipoprotein cholesterol (LDL-C) levels in a subject to whom a described ANGPTL3 RNAi agent has been administered is reduced by at least about 5%, 10%, 15%, 20%, 25%, 30%, 35%, or greater than 35% relative to the subject prior to being administered the ANGPTL3 RNAi agent or to a subject not receiving the ANGPTL3 RNAi agent. The LDL-C level in the subject is reduced in the blood/serum of the subject, and can be calculated using methods known in the art such as the Martin-Hopkins method (See. e.g., H. Ginsberg et al., LDL-C calculated by Friedewald, Martin-Hopkins, or NIH equation 2 versus beta-quantification: pooled alirocumab trials, J. Lipid Research 63 (1) (January 2022)).

In some embodiments, non-HDL cholesterol levels in a subject to whom a described ANGPTL3 RNAi agent has been administered is reduced by at least about 5%, 10%, 15%, 20%, 25%, 30%, 35%, 40%, or greater than 40% relative to the subject prior to being administered the ANGPTL3 RNAi agent or to a subject not receiving the ANGPTL3 RNAi agent. The non-HDL cholesterol levels in the subject is reduced in a cell, group of cells, tissue, blood, and/or other fluid of the subject. In some instances, serum concentrations of non-HDL cholesterol levels are assessed.

In some embodiments, the apolipoprotein B (ApoB) levels in a subject to whom a described ANGPTL3 RNAi agent has been administered is increased by at least about 5%, 10%, 15%, 20%, 25%, 30%, or greater than 30% relative to the subject prior to being administered the ANGPTL3 RNAi agent or to a subject not receiving the ANGPTL3 RNAI agent. The ApoB levels in the subject is reduced in a cell, group of cells, tissue, blood, and/or other fluid of the subject. In some instances, serum concentrations of ApoB are assessed.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition of the number, severity, and/or frequency of one or more symptoms of a disease in a subject. As used herein, unless otherwise expressly noted, the phrase “treatment of a disease or disorder” includes the treatment of underlying conditions and/or symptoms of the disease, as would be understood by the person of ordinary skill in the art. Unless otherwise expressly limited herein, such phrases are not intended to be limiting.

As used herein, an “ANGPTL3-related disease or disorder” refers to any disease or disorder that can be treated at least in part by a reduction or inhibition in ANGPTL3 gene expression using the ANGPTL3 RNAi agent, including by the methods of treatment described herein. As discussed herein, the methods of treatment disclosed herein, among other things, result in a reduction in TG levels in a subject to which the ANGPTL3 RNAi Drug Substance is administered. ANGPTL3-related diseases and disorders include, but are not limited to, hypertriglyceridemia (including severe hypertriglyceridemia (SHTG)), homozygous familial hypercholesterolemia (HoFH), hypertriglyceridemia induced pancreatitis, metabolic syndrome, obesity, hyperlipidemia, mixed dyslipidemia, abnormal lipid and/or cholesterol metabolism, atherosclerosis, cardiovascular disease, type II diabetes mellitus, coronary artery disease, non-alcoholic steatohepatitis, non-alcoholic fatty liver disease, heterozygous familial hypercholesterolemia (HeFH), statin resistant hypercholesterolemia, other dyslipidemias, and other metabolic-related disorders and diseases, lipodystrophy syndromes including familial partial lipodystrophy, obesity, dyslipidemia, non-alcoholic steatohepatitis, non-alcoholic fatty liver disease, familial chylomicronemia syndrome (FCS), chylomicronemia, multifactorial chylomicronemia, and other dyslipidemias and metabolic-related disorders and diseases.

As used herein, “monthly dosing” or “monthly” administration means every 28 days. As used herein, “quarterly dosing” or “quarterly” administration means every 84 days and unless expressly noted otherwise is synonymous with dosing every 12 weeks (q12w). The term “about” when used in connection with monthly dosing means monthly dosing+/−5 days. The term “about” when used in connection with quarterly dosing means quarterly dosing+/−14 days.

As used herein, the phrase “introducing into a cell,” when referring to an RNAi agent, means functionally delivering the RNAi agent into a cell. The phrase “functional delivery,” means that delivering the RNAi agent to the cell in a manner that enables the RNAi agent to have the expected biological activity, e.g., sequence-specific inhibition of gene expression.

Unless stated otherwise, use of the symbol [symbol] as used herein means that any group or groups may be linked thereto that is in accordance with the scope of the inventions described herein.

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below: All publications, patent applications, patents, and other references mentioned herein are incorporated by reference in their entirety. In case of conflict, the present specification, including definitions, will control. In addition, the materials, methods, and examples are illustrative only and not intended to be limiting.

The above provided embodiments and items are now illustrated with the following, non-limiting examples.

### EXAMPLES

### Example 1. Synthesis and Formulation of ANGPTL3 RNAi Drug Substance (ADS-003)

The ANGPTL3 RNAi Drug Substance suitable for use in the methods disclosed herein can be synthesized using standard phosphoramidite technology on solid phase oligonucleotide synthesis as is known in the art. Commercially available oligonucleotide synthesizers (e.g., MerMade96E® (Bioautomation) or MerMade12® (Bioautomation)) may be used. Syntheses can be performed on a solid support made of controlled pore glass (CPG, 500 Å or 600 Å, obtained from Prime Synthesis, Aston, PA, USA). The monomer positioned at the 3′ end of the respective strand may be attached to the solid support as a starting point for synthesis. All RNA, 2′-modified RNA phosphoramidites, and inverted abasic phosphoramidites can be purchased commercially. Targeting group-containing phosphoramidites can be synthesized that are suitable for addition to the 5′ end of the sense strand. Standard cleavage, deprotection, purification, and annealing steps can be utilized as is known in the art. Further description related to the synthesis of ANGPTL3 RNAi agents (including the targeting group-containing phosphoramidites) may be found, for example, in International Patent Application Publication No. WO 2019/055633 (Patent Application No. PCT/US2018/050848) and WO 2018/044350 (PCT/US2017/021147), each of which is incorporated by reference herein in its entirety. ANGPTL3 RNAi Drug Substance can then be formulated by dissolving in standard pharmaceutically acceptable excipients that are generally known in the art. For example, Table 3 shows a Formulated ANGPTL3 RNAI Drug Substance that is suitable for use in the methods disclosed herein. The Formulated ANGPTL3 RNAi Drug Substance was prepared at a commercial GMP fill/finish manufacturing facility.

### Example 2. Phase 2b Clinical Trial to Evaluate the Efficacy and Safety of ANGPTL3 RNAi Drug Substance (ADS-003) in Adults with Mixed Dyslipidemia

A Phase 2b study to evaluate the efficacy and safety of ANGPTL3 RNAi Drug Substance (ADS-003) in adults with mixed dyslipidemia was initiated.

Three dose levels of ANGPTL3 RNAi Drug Substance (ADS-003) are to be evaluated against placebo in participants with mixed dyslipidemia that had fasting TG levels between 150-499 mg/dL, and either (i) low-density lipoprotein cholesterol (LDL-C)≥70 mg/dL (1.8 mmol/L); or (ii) non-high-density lipoprotein cholesterol (non-HDL-C)≥100 mg/dL (2.59 mmol/L).

A total of approximately 180 participants were planned to be enrolled in the study: however, the study over-enrolled and at least 203 patients entered the study. All dose cohorts were enrolled in parallel and subjects were randomly assigned in a 3:1 active: placebo ratio to receive (i) ANGPTL3 RNAi Drug Substance (ADS-003) (consisting of the Formulated ANGPTL3 RNAi Drug Substance as described in Table 3), or (ii) placebo. During the double blind treatment period, each participant will receive a subcutaneous (SC) injection on Day 1 and again on Week 12 for a total of 2 injections as follows:
ARO-ANG3 50 mg (n=45*) or volume-matched placebo (n=15*) at Day 1 and Week 12; or
ARO-ANG3 100 mg (n=45*) or volume-matched placebo (n=15*) at Day 1 and Week 12; or
ARO-ANG3 200 mg (n=45*) or volume-matched placebo (n=15*) at Day 1 and Week 12.
* As noted above, the study over-enrolled and thus additional subjects were added to the groups in parallel in the same 3:1 ratio.

As used herein, ARO-ANG3 is interchangeable with ANGPTL3 RNAi Drug Substance (ADS-003) in the delivery form of the Formulated ANGPTL3 RNAi Drug Substance as described in Table 3.

After completing the Week 36 visit in the double blind treatment period, participants may opt to continue in a 24-month open-label extension (OLE) treatment period. Participants in the OLE treatment period will be dosed quarterly and will be assigned to receive the same dose level to which they were randomized in the double blind treatment period. Once a single dose has been selected for future clinical studies, all participants will be transitioned to the selected dose level.

TABLE 4
Clinical Study Parameters

| Development Phase | Phase 2b | |
|---|---|---|
| Study Objectives | Primary Objectives: | |
| | To evaluate the safety and efficacy of ANGPTL3 RNAi Drug | |
| | Substance (ADS-003) in adults with mixed dyslipidemia and | |
| | to select a dosing regimen for later stage clinical studies in | |
| | this patient population. Further, to evaluate the efficacy, | |
| | safety, and tolerability of long-term dosing of ANGPTL3 | |
| | RNAi Drug Substance (ADS-003) in a 24-month OLE | |
| | treatment period following the double blind treatment period. | |
| Endpoints | Primary Endpoint: | |
| | The primary endpoint of the study is: | |
| | Percent change from baseline at Week 24 in fasting TG. | |
| | Secondary Endpoints: | |
| | The following secondary endpoints will be evaluated during | |
| | 36-week double-blind study period: | |
| | Percent change from baseline in fasting TG over time | |
| | through Week 36; | |
| | Percent change from baseline at Week 24 and over time | |
| | through Week 36 in fasting non--HDL-C; | |
| | Percent change from baseline at Week 24 and over time | |
| | through Week 36 in fasting total apolipoprotein B | |
| | (ApoB); | |
| | Percent change from baseline at Week 24 and over time | |
| | through Week 36 in fasting LDL-C using | |
| | ultracentrifugation; | |
| | Percent change from baseline at Week 24 and over time | |
| | through Week 36 in angiopoietin-like protein 3 | |
| | (ANGPTL3); | |
| | Percent change from baseline at Week 24 and over time | |
| | through Week 36 in fasting HDL-C; | |
| | Plasma concentrations of ARO-ANG3 over time through | |
| | Week 12; and | |
| | The frequency and severity of AEs (Adverse Events) and | |
| | SAEs (Severe Adverse Events) at Week 24 and over time | |
| | through Week 36. | |
| | The following secondary endpoints will be evaluated in | |
| | the OLE treatment period: | |
| | Percent change from baseline in fasting TG, non-HDLC, | |
| | total ApoB, LDL-C using ultracentrifugation, | |
| | ANGPTL3, HDL-C, and plasma ARO-ANG3. | |
| | The frequency and severity of Adverse Events and | |
| | Severe Adverse Events through Month 24. | |
| | Exploratory Endpoints: | |
| | The following exploratory endpoints will be evaluated | |
| | in this study: | |
| | Change from baseline over time during double-blind | |
| | study period as well as over time during the OLE | |
| | treatment period in other fasting lipid parameters (total | |
| | cholesterol, LDL/HDL ratio, very low-density | |
| | lipoprotein-cholesterol [VLDL-C], ApoB-48, lipoprotein | |
| | [LP]a, ApoB-100, ApoC-III, ApoC-II, ApoA-I, ApoA-V, | |
| | and ApoA-1) | |
| | Change from baseline to Week 24 and over time through | |
| | Week 36 during double-blind study period as well over | |
| | time in the OLE treatment period in fasting serum blood | |
| | glucose, HbA_{1c}, homeostatic model assessment for | |
| | insulin resistance (HOMA-IR) and C-peptide | |
| | Proportion of participants requiring emergent apheresis | |
| | over time during double-blind study period as well as | |
| | during the OLE treatment period | |
| | Change from baseline to Week 24 in liver fat content | |
| | using magnetic resonance imaging-proton density fat | |
| | fraction (MRI-PDFF; only in participants with a liver fat | |
| | fraction of ≥8% at Screening); and | |
| | Emergence of and levels of anti-drug antibodies to ARO- | |
| | ANG3 in those receiving AROANG3 over time during | |
| | double-blind study period as well as over time during the | |
| | OLE treatment period. | |
| Study Design | Randomized, double-blind, placebo-controlled, Phase 2b clinical | |
| | study with an OLE treatment period | |
| Study Population/ | To be eligible for enrollment, participants must meet all the | |
| Inclusion Criteria | following inclusion criteria: | |
| | 1. | Males or nonpregnant (who do not plan to become |
| | | pregnant), nonlactating females ≥18 years of age; |
| | 2. | Based on medical history, prior evidence of TG ≥150 |
| | | mg/dL (≥1.69 mmol/L) but ≤499 mg/dL (5.61 |
| | | mmol/L); |
| | 3. | Fasting levels at Screening of LDL-C ≥70 mg/dL |
| | | (1.8 mmol/L) OR non-HDL-C ≥100 mg/dL |
| | | (2.59 mmol/L) after at least 4 weeks of stable |
| | | diet and stable optimal statin therapy (unless |
| | | documented as statin intolerant); |
| | 4. | A mean fasting TG level of ≥150 mg/dL |
| | | (≥1.69 mmol/L) and ≤499 mg/dL (5.61 mmol/L) |
| | | collected at two separate and consecutive visits and at |
| | | least 7 days apart and no more 17 days apart during the |
| | | Screening period; |
| | 5. | Able and willing to provide written informed consent |
| | | prior to the performance of any study specific procedures; |
| | 6. | Willing to follow diet counseling and maintain a stable |
| | | diet as per investigator judgment based on local standard |
| | | of care; |
| | 7. | Participants of childbearing potential must agree to use |
| | | highly-effective contraception, during the study and for at |
| | | least 24 weeks from last dose of the Investigational |
| | | Product (IP). Males must not donate sperm during the |
| | | study and for at least 24 weeks following the last dose of |
| | | IP; |
| | 8. | Women of childbearing potential on hormonal |
| | | contraceptives must be stable on the medication |
| | | for ≥2 menstrual cycles prior to Day 1; |
| | 9. | Participants must be on at least 4 weeks of stable optimal |
| | | statin therapy (unless statin intolerant). Participants |
| | | taking any of the following medications must be on a |
| | | stable regimen for the specified duration prior to |
| | | collection of Screening visit (S2) laboratory tests and for |
| | | the duration of study participation. |
| | | Time on stable regimen |
| | | prior to collection of |
| | | Screening visit (S2) |
| | Medication | laboratory tests |
| | Lipid lowering therapies | ≥4 weeks |
| | (including statins) | |
| | Beta-blockers, thiazide diuretics | ≥4 weeks |
| | Fibrates | ≥6 weeks |
| | PCSK9 inhibitors | ≥8 weeks |
| | Retinoids | ≥8 weeks |
| | Atypical antipsychotics | ≥12 weeks |
| | Diabetes mellitus medications | ≥12 weeks |
| | Anticoagulation therapy | ≥12 weeks |
| | Thyroid hormone replacement | ≥12 weeks |
| | Testosterone replacement therapy | ≥16 weeks |
| | Oral estrogens, tamoxifen, | ≥16 weeks |
| | Immunosuppressants | ≥24 weeks |
| | Exclusion Criteria: | |
| | 1. | Current use or use within the last 365 days from Day 1 of |
| | | any hepatocyte targeted RNAi agent or antisense |
| | | oligonucleotide molecule; |
| | 2. | Active pancreatitis within 12 weeks prior to Day 1; |
| | 3. | Any planned bariatric surgery or similar procedures to |
| | | induce weight loss during the period starting at consent |
| | | through the end of the study; |
| | 4. | History of major surgery within 12 weeks of Day 1 or |
| | | planned major surgery during the study; |
| | 5. | Planned coronary intervention (such as stent placement or |
| | | heart bypass) during the study; |
| | 6. | History of acute coronary syndrome event within 24 |
| | | weeks of Day 1; |
| | 7. | New York Heart Association (NYHA) Class II, III, or IV |
| | | heart failure or last known ejection fraction of <30%; |
| | 8. | Uncontrolled hypertension (sitting blood pressure >160/100 |
| | | mmHg at Screening), participant may be re- |
| | | screened once hypertension is controlled; |
| | 9. | History of hemorrhagic stroke within 24 weeks of Day 1; |
| | 10. | History of bleeding diathesis or coagulopathy; |
| | 11. | Current diagnosis of nephrotic syndrome; |
| | 12. | Any of the following laboratory values at Screening: |

| | |
|---|---|
| a. | Hepatic: ALT or AST >2 × ULN at Screening; |
| b. | Estimated glomerular filtration rate (eGFR) <30 |
| | mL/min/1.73 m² (using the Modification of Diet |
| | in Renal Disease [MDRD] equation) at |
| | Screening; |
| c. | Glycosylated hemoglobin (HbA_{1c}) >9.0% |
| | (or >75 mmol/mol International Federation of |
| | Clinical Chemistry [IFCC] units) at Screening; |
| d. | Spot urine protein/spot urine creatinine ratio >3 |
| | grams per day; |
| e. | Clinically significant abnormality in PT, aPTT, or |
| | INR; |

| | |
|---|---|
| 13. | Systemic use of corticosteroids or anabolic steroids |
| | within 4 weeks prior to Day 1 or planned use during the |
| | study (stable doses of testosterone replacement therapy >16 |
| | weeks prior to Screening (visit S2) is permitted for a |
| | documented history of hypogonadism [low testosterone] |
| | as verified in subject health records); |
| 14. | Blood donation of 50 to 499 mL within 4 weeks of |
| | Screening (visit S2) or of >499 mL within 8 weeks of |
| | Screening (visit S2) laboratory collection; |
| 15. | Known history of human immunodeficiency virus |
| | infection; |
| 16. | Seropositive (hepatitis B surface antigen [HBsAg] +) for |
| | hepatitis B virus (HBV) or hepatitis C virus (HCV) (HCV |
| | seropositivity requires positive test for antibodies |
| | confirmed with positive test for HCV RNA); |
| 17. | Clinical evidence of uncontrolled hypothyroidism or |
| | hyperthyroidism as per Investigator's judgment; |
| 18. | History of malignancy within the last 2 years prior to the |
| | date of consent requiring systemic treatment except for |
| | adequately treated basal cell carcinoma, squamous cell |
| | skin cancer, superficial bladder tumors, or in situ cervical |
| | cancer. Currently receiving systemic cancer treatment(s) |
| | or, in the Investigator's opinion, at risk of relapse for |
| | recent cancer; |
| 19. | Use of an investigational agent or device within 30 days |
| | or within 5 half-lives, based on plasma pharmacokinetics |
| | (PK) (whichever is longer) prior to Day 1 or current |
| | participation in an interventional investigational study. |
| | Participants previously exposed to ARO-ANG3 or ARO- |
| | APOC3 will require a washout period of at least 1 year |
| | from last dose; or |
| 20. | Unwilling to limit alcohol consumption to within |
| | moderate limits for the duration of the study, as follows: |
| | not more than 14 units per week (1 unit = 80 mL of wine, |
| | 200 mL of beer, or 25 mL of 40% alcohol); |
| 21. | Any concomitant medical, psychiatric condition, social |
| | situation, or any other situation that, in the Investigator's |
| | judgment, would make it difficult to comply with |
| | protocol requirements or put the participant at additional |
| | safety risk. |

| | |
|---|---|
| | All laboratory tests used as exclusion criteria may be repeated |
| | once and the repeat value may be used for exclusion purposes. |
| | A participant will be excluded from the OLE treatment period of |
| | the study if any new conditions or worsening of any existing |
| | condition (e.g., renal, hematologic, gastrointestinal, endocrine, |
| | cardiovascular, pulmonary, immunologic, psychiatric) or any |
| | other situation that, in the investigator's judgment, would make |
| | the subject unsuitable for enrollment, or which could otherwise |
| | interfere with the subject participating in or completing the study, |
| | or would make it difficult to comply with protocol requirements |
| | or put the participant at additional safety risk |
| Investigational | ANGPTL3 RNAi Drug Substance (ADS-003) (see Table 2), |
| Product (IP) | administered as Formulated ANGPTL3 RNAi Drug Substance |
| | (see Table 3) |
| Dosage and | Subjects were randomized to receive ANGPTL3 RNAi Drug |
| Frequency | Substance (ADS-003) or placebo (3 active: 1 placebo) within each |
| | dosing cohort at a dose of 50 mg, 100 mg, or 200 mg, |
| | administered as a subcutaneous injection on day 1 and again at |
| | week 12. |
| Reference | Placebo (PBO): normal saline (0.9%) administered |
| Formulation | subcutaneously with matching volume. |
| Data Analysis | Efficacy and Pharmacodynamics Analysis: |
| | The primary endpoint will be evaluated using mixed model |
| | repeated measures approach with a minimum model terms |
| | included for treatment group, baseline value, visit, and |
| | stratification factors. The primary analysis will be performed will |
| | evaluate the difference in means between each ARO-ANG3 dose |
| | group and pooled placebo groups in all randomized participants |
| | who receive at least 1 dose of IP (Full Analysis Set). The primary |
| | estimand of interest is the difference in means of percent change |
| | from baseline in fasting TG at Week 24 during double-blind study |
| | period in adult Mixed Dyslipidemia population (as defined by the |
| | inclusion/exclusion criteria), regardless of treatment compliance |
| | or other intercurrent events postbaseline. When performing the |
| | primary analysis, the adjustment for multiplicity of testing several |
| | arms versus placebo will be carried out using Holm's step-down |
| | procedure. |
| | All continuous secondary endpoints of double-blind study period |
| | will be analyzed in a similar manner to the primary endpoint, |
| | unless otherwise noted. |
| | Data will be summarized as applicable for the following: Serum |
| | ANGPTL3, ratio of LDL-C/HDL-C, ApoC-III, ApoC-II, LDL-C, |
| | total cholesterol, non-HDL-C, HDL-C, VLDL-C, LP(a), TG, |
| | ApoB-48, ApoB-100, total ApoB, ApoA-I, ApoA-V, HbA_{1c}, C- |
| | peptide, HOMA-IR, changes in serum insulin, and changes in |
| | serum glucose. |
| | For primary endpoint (TG) analysis at Week 24 during double- |
| | blind study period, the laboratory value for endpoint analysis will |
| | be the arithmetic mean of 2 values taken during Week 24. If only |
| | 1 value is available during Week 24, then this value will be used |
| | for endpoint analysis. For data analysis purposes, baseline will be |
| | defined as the arithmetic mean of Day 1 predose assessment and |
| | two fasting assessments during screening period. For other |
| | efficacy endpoints, only Day 1 predose assessment will be used as |
| | the baseline. |
| | After the last subject in the double blind treatment period |
| | completes the Week 36 Visit, an interim analysis will be |
| | conducted to review the efficacy and safety data in order to select |
| | a single dose level for all subjects in OLE treatment period and |
| | for the remainder of their participation in the trial. |
| | For efficacy variables during open-label treatment period, the |
| | measurements and changes and/or percent changes from baseline |
| | over time will be summarized descriptively. |
| | Descriptive statistics of biomarker changes will include mean, |
| | median SD, minimum, and maximum. Additional details will be |
| | provided in the statistical analysis plan. |
| | Tolerability and Safety Data: |
| | In general, safety analyses will be performed and the results |
| | summarized by dose cohort. Post-treatment safety assessments |
| | will be compared with measurements recorded at baseline. |
| | Treatment emergent AEs will be summarized using the latest |
| | version of the Medical Dictionary for Regulatory Activities |
| | (MedDRA) by System Organ Class (SOC) and Preferred Term |
| | (PT). The incidence and frequency of AEs, SAEs, related AEs, |
| | related SAEs, and AEs leading to discontinuation, will be |
| | summarized by dose cohort per SOC, PT, and severity. All AEs |
| | will also be presented in listings. The duration of AEs will be |
| | determined and included in listings, along with the action taken |
| | and outcome. The incidence of laboratory abnormalities will be |
| | assessed using descriptive summary statistics and shift tables. |
| | Vital sign measurements will be summarized at each scheduled |
| | time point using descriptive statistics. Abnormal physical |
| | examination findings will be summarized by time point and |
| | presented in participant listings. ECG parameters, changes from |
| | baseline, and qualitative assessments will be summarized. |
| | Pregnancy and follicle-stimulating hormone (FSH) test results |
| | will be listed separately by time point. |
| | All safety and tolerability analyses will be performed using the |
| | Safety Analysis Population, defined as all enrolled participants |
| | who received at least 1 dose of IP. |
| | Pharmacokinetics (PK): |
| | Plasma concentrations of ARO-ANG3 will be measured in all |
| | participants to evaluate trough and postdose levels throughout the |
| | study. |
| | Full PK group: In each of the dose cohorts, approximately 16 PK |
| | participants (12 active, 4 placebo for 48 total participants for PK) |
| | will be enrolled at the designated PK sites. Plasma concentrations |
| | will be measured predose, serially postdose on Day 1 and Week |
| | 12. Only samples collected from participants receiving active |
| | treatment will be included in the PK analysis. |
| | Sparse Sample PK group: All participants will provide a predose |
| | and postdose PK sample on Day 1 and Week 12. |
| | Immunogenicity (Anti-drug Antibodies): |
| | Changes from assay negative to positive will be summarized by |
| | dose and number of doses administered. Descriptive statistics of |
| | immunogenicity parameters will include mean, SD, minimum, |
| | and maximum. |

### Interim Results (Data Cutoff 6 Jul. 2022)

As noted above, eligible subjects (n=203) were randomized 3:1 to receive subcutaneous injections of 50, 100, or 200 mg ARO-ANG3 or placebo on Day I and at Week 12. Subjects were required to be on a stable diet and optimal statin/lipid-lowering therapies. In subjects with hepatic steatosis, liver fat was assessed at baseline and Week 24 by magnetic resonance imaging-derived proton density fat fraction (MRI-PDFF). At the time of the interim analysis all subjects reached Week 12. Serum lipid and lipoprotein levels, including LDL-C(Martin Hopkins), were determined at Week 16 after most subjects had received both doses.

At Week 16, ARO-ANG3 dose-dependently decreased median TGs (by 53% to 59%) and reduced mean atherogenic lipoproteins including LDL-C(by 22% to 32%), non-HDL-C(by 28% to 36%), apolipoprotein B (ApoB; by 13% to 22%), remnant cholesterol (by 40% to 47%), and mean HDL-C was reduced by 17% to 31%. Among 54 subjects (40 ARO-ANG3, 14 placebo) with hepatic steatosis, median percent changes from baseline at Week 24 in liver fat ranged from −15% (50 mg) to −28% (200 mg) with ARO-ANG3 compared to-6% for placebo. Because the study remains blinded, safety data were pooled regardless of treatment assignment, and 135/203 (67%) subjects had greater than or equal to 24 weeks of exposure. The most frequent adverse events were COVID-19 (11%), urinary tract infection (8%), headache (6%), upper respiratory infection (6%), and injection site pain (5%). Data are further summarized in the following Table 5:

TABLE 5
Percent Change from Baseline in Serum Lipid
and Lipoprotein Concentrations at Week 16

| Serum Concentration | | | |
|---|---|---|---|
| Pooled | ARO-ANG3 | | |
| Placebo | 50 mg | 100 mg | 200 mg |
| (n = 51) | (n = 51) | (n = 50) | (n = 51) |

TG

| | | | | |
|---|---|---|---|---|
| Baseline median, | 219.9 | 223.3 | 231.2 | 234.1 |
| mg/dL | (163.2, 266.8) | (173.8, 303.3) | (190.5, 265.4) | (183.5, 326.2) |
| (Q1, Q3) | | | | |
| Median % change at | 7.2 | −52.9 | −56.4 | −59.1 |
| Week 16 (Q1, Q3) | (−21.4, 25.2) | (−66.5, −35.8) | (−64.3, −44.0) | (−71.0, −49.8) |
| | (n = 49) | (n = 48) | (n = 46) | (n = 51) |

LDL-C (Martin-Hopkins)

| | | | | |
|---|---|---|---|---|
| Baseline mean, mg/dL | 102.5 (30.6) | 112.8 (29.7) | 108.7 (44.8) | 105.6 (33.7) |
| (SD) | | | | |
| Mean % change at | 5.3 (45.6) | −23.2 (24.4) | −22.1 (24.3) | −31.7 (23.3) |
| Week 16 (SD) | (n = 49) | (n = 48) | (n = 46) | (n = 51) |

Non-HDL-C

| | | | | |
|---|---|---|---|---|
| Baseline mean, mg/dL | 138.6 (41.6) | 151.5 (36.0) | 149.3 (47.5) | 143.3 (39.6) |
| (SD) | | | | |
| Mean % change at | 4.2 (26.2) | −28.0 (19.9) | −30.8 (17.0) | −36.3 (17.8) |
| Week 16 (SD) | (n = 49) | (n = 48) | (n = 46) | (n = 51) |

ApoB

| | | | | |
|---|---|---|---|---|
| Baseline mean, mg/dL | 95.7 (24.1) | 106.8 (23.4) | 99.6 (26.2) | 94.9 (25.0) |
| (SD) | | | | |
| Mean % change at | 0.0 (22.1) | −18.8 (16.3) | −13.2 (17.5) | −21.8 (21.3) |
| Week 16 (SD) | (n = 30) | (n = 29) | (n = 30) | (n = 32) |

Remnant Cholesterol

| | | | | |
|---|---|---|---|---|
| Baseline mean, mg/dL | 36.1 (31.6) | 38.7 (12.1) | 40.6 (30.8) | 37.6 (14.9) |
| (SD) | | | | |
| Mean % change at | 21.9 (93.6) | −40.4 (18.4) | −43.4 (16.6) | −46.8 (14.5) |
| Week 16 (SD) | (n = 49) | (n = 48) | (n = 46) | (n = 51) |

HDL-C

| | | | | |
|---|---|---|---|---|
| Baseline mean, mg/dL | 41.6 (11.9) | 43.2 (13.3) | 39.9 (10.6) | 42.3 (13.6) |
| (SD) | | | | |
| Mean % change at | 2.1 (16.0) | −17.3 (26.3) | −28.6 (20.9) | −30.6 (24.1) |
| Week 16 (SD) | (n = 49) | (n = 48) | (n = 46) | (n = 51) |

Further, in subjects with mixed dyslipidemia who had baseline median TGs of 220 mg/dL to 234 mg/dL, treatment with ARO-ANG3 at doses of 50 mg, 100 mg or 200 mg ARO-ANG3 resulted in substantial reductions of ANGPTL3 (up to 71% at Week 8), TGs (up to 59% at Week 16); and LDL-C(up to 32% at Week 16).

ARO-ANG3 was also shown to be associated with relative reduction in liver fat fraction at Week 24 (approximately 30% relative reduction in liver fat (MRI-PDFF)), with no AEs related to Liver Function Test (LFT) changes reported to date. TEAEs reported to date are consistent with those expected in this patient population and with associated underlying comorbidities. The favorable changes in serum lipids and lipoproteins support the potential value of ARO-ANG3 for the treatment of mixed dyslipidemia in patients at risk of atherosclerotic cardiovascular disease, as well as patients with familiar hypercholesterolemia and/or metabolic syndrome. Indications for the treatment of patients with homozygous familial hypercholesterolemia (HoFH) and heterozygous familial hypercholesterolemia (HeFH) appears particularly promising in view of the data from the Phase 2b clinical study. The data also suggest that dosing once every approximately three months or approximately every twelve weeks (q12w or q3m) seems feasible and may lead to improved patient adherence.

### Updated Results (Data Cutoff 9 Dec. 2022)

Data were further evaluated at end-of-study. The results were consistent with the reported interim results described immediately above.

At Week 24, ARO-ANG3 decreased ANGPTL3 protein (up to 77%), TGs (up to 56%), remnant cholesterol (up to 56%), and apolipoprotein B (apoB) (up to 20%). Other lipoproteins including LDL C, non-HDL-C, lipoprotein (a) and HDL-C were also decreased. Liver fat decreased similarly in ARO-ANG3 and placebo groups. Among 58 subjects with hepatic steatosis, median percent changes from baseline at Week 24 in liver fat ranged from −16% (50 mg) to −29% (200 mg) with ARO-ANG3 compared to −19% for placebo. TEAEs reported to date are consistent with those expected in this patient population and with associated underlying comorbidities. The most frequent adverse events were COVID-19 (20%), upper respiratory infection (7%), urinary tract infection (7%), headache (7%), and injection-site pain (5%).

TABLE 5B
Percent Change from Baseline in Serum Lipid and Lipoprotein Concentrations at Week 24
Percent Change from Baseline in Serum Lipid and Lipoprotein Concentrations at Week 24

| Serum Concentration | | | |
|---|---|---|---|
| Pooled | ARO-ANG3 | | |
| Placebo | 50 mg | 100 mg | 200 mg |
| (n = 51) | (n = 51) | (n = 51) | (n = 51) |

TG

| Baseline median, | 219.9 | | 223.3 | | 228.4 | | 234.1 | |
|---|---|---|---|---|---|---|---|---|
| mg/dL | (134.8, 600.1) | | (134.7, 502.1) | | (152.4, 674.8) | | (112.4, 549.4) | |
| (Min, Max) | | | | | | | | |
| LS mean (SE) % | 7.6 | (3.8) | −43.7 | (3.8) | −49.0 | (3.8) | −55.6 | (3.7) |
| change at Week 24 | | | | | | | | |
| LS mean of difference | — | | −51.2** | | −56.6** | | −63.1** | |
| vs placebo | | | | | | | | |

LDL-C (Ultracentrifugation)⁺⁺

| Baseline mean, | 93.7 | (31.2) | 102.8 | (29.4) | 102.8 | (44.2) | 92.1 | (34.1) |
|---|---|---|---|---|---|---|---|---|
| mg/dL (SD) | | | | | | | | |
| LS mean (SE) % | 4.7 | (4.0) | −11.1 | (4.0) | −8.9 | (4.1) | −15.2 | (4.0) |
| change at Week 24 | | | | | | | | |
| LS mean of difference | — | | −15.8* | | −13.6* | | −19.9* | |
| vs placebo | | | | | | | | |

Non-HDL-C

| Baseline mean, | 138.6 | (41.6) | 151.3 | (36.2) | 149.8 | (47.2) | 143.3 | (39.6) |
|---|---|---|---|---|---|---|---|---|
| mg/dL (SD) | | | | | | | | |
| LS mean (SE) % | 4.2 | (3.3) | −25.1 | (3.3) | −24.6 | (3.3) | −32.2 | (3.3) |
| change at Week 24 | | | | | | | | |
| LS mean of difference | — | | −29.2** | | −28.7** | | −36.4** | |
| vs placebo | | | | | | | | |

ApoB

| Baseline mean, | 95.6 | (24.4) | 105.0 | (24.1) | 100.1 | (25.8) | 94.1 | (25.0) |
|---|---|---|---|---|---|---|---|---|
| mg/dL (SD) | | | | | | | | |
| LS mean (SE) % | 2.3 | (2.8) | −16.4 | (2.8) | −12.9 | (2.8) | −19.6 | (2.8) |
| change at Week 24 | | | | | | | | |
| LS mean of difference | — | | −18.7** | | −15.2* | | −21.9** | |
| vs placebo | | | | | | | | |

Remnant Cholesterol

| Baseline mean, | 44.9 | (34.3) | 48.5 | (19.2) | 48.6 | (31.4) | 51.2 | (27.5) |
|---|---|---|---|---|---|---|---|---|
| mg/dL (SD) | | | | | | | | |
| LS mean (SE) % | 25.9 | (7.7) | −46.7 | (7.7) | −50.0 | (7.8) | −56.1 | (7.6) |
| change at Week 24 | | | | | | | | |
| LS mean of difference | — | | −72.6** | | −75.9** | | −82.0** | |
| vs placebo | | | | | | | | |

HDL-C

| | | | | | | | | |
|---|---|---|---|---|---|---|---|---|
| Baseline mean, | 41.6 | (11.9) | 43.1 | (13.2) | 39.8 | (10.5) | 42.3 | (13.6) |
| mg/dL (SD) | | | | | | | | |
| LS mean (SE) % | 3.1 | (2.9) | −9.0 | (2.9) | −18.5 | (3.0) | −21.5 | (2.9) |

| change at Week 24 | | | | |
|---|---|---|---|---|
| LS mean of difference | — | −12.0* | −21.6** | −24.5** |
| vs placebo | | | | |

Lipoprotein(a)

| Baseline mean, | 97.0 | (187.3) | 64.0 | (111.0) | 91.3 (118.8) | 63.3 | (95.5) |
|---|---|---|---|---|---|---|---|
| nmol/L | | | | | | | |
| LS mean (SE) % | 8.3 | (5.3) | 1.0 | (5.4) | −11.7 (5.3) | −8.8 | (5.3) |
| change at Week 24 | | | | | | | |
| LS mean of difference | — | | −7.34 | | −20.0* | −17.1* | |
| vs placebo | | | | | | | |

⁺⁺One patient with baseline value at 17 mg/dL was removed from the analysis;
*p < 0.05;
**p < 0.0001

### Example 3. Phase 1 Single and Multiple Dose Study to Evaluate the Safety, Tolerability, Pharmacokinetics, and Pharmacodynamic Effects of ANGPTL3 RNAI Drug Substance (ADS-003) in Adult Healthy Volunteers and in Dyslipidemic Patients

A Phase 1 single and multiple dose study to evaluate the safety, tolerability, pharmacokinetics, and pharmacodynamics of ANGPTL3 RNAi Drug Substance (ADS-003) in normal healthy volunteer adults (NHVs) and in adults with dyslipidemia was conducted.

Several dose levels of ANGPTL3 RNAi Drug Substance (ADS-003) were evaluated against placebo (PBO) in accordance with the following cohort dosing summary:

TABLE 6
Cohort Summary for Phase 1 Clinical Study

| Cohort | Population | Blinding | # Subjects | Dosing Schedule |
|---|---|---|---|---|
| 1 | NHVs TGs >100 mg/dL(1.13 | Double- | 10 (6 active: | 35 mg on Day 1 only |
| | mmol/L), | blind | 4 PBO) | |
| | LDL-C >70 mg/dL (1.81 mmol/L) | | | |
| 2 | NHVs TGs >100 mg/dL (1.13 | Double- | 10 (6 active: | 100 mg Day 1 only |
| | mmol/L), | blind | 4 PBO) | |
| | LDL-C >70 mg/dL(1.81 mmol/L) | | | |
| 2b | NHVs | Open-label | 4 active | 100 mg Day 1, 29 |
| 3 | NHVs TGs >100 mg/dL (1.13 | Double- | 10 (6 active: | 200 mg Day 1 only |
| | mmol/L), | blind | 4 PBO) | |
| | LDL-C >70 mg/dL(1.81 mmol/L) | | | |
| 3b | NHVs | Open-label | 4 active | 200 mg Day 1, 29 |
| 4 | NHVs TGs >100 mg/dL (1.13 | Double- | 10 (6 active: | 300 mg Day 1 only |
| | mmol/L), | blind | 4 PBO) | |
| | LDL-C >70 mg/dL (1.81 mmol/L) | | | |
| 4b | NHVs | Open-label | 4 active | 300 mg Day 1, 29 |
| 5 | NHVs with liver fat on | Double- | 9 (6 active: 3 | 200 mg Day 1, 29 |
| | MRI-PDFF ≥10% | Blind | PBO) | |
| 6 | LDL-C >70 mg/dL (1.81 mmol/L) | Double- | 9 (6 active: 3 | 200 mg Day 1, 29 |
| | on stable statin regimen | Blind | PBO) | |
| 7 | Familial Hypercholesterolemia | Open-label | ≤6 active | 200 mg Day 1, 29 |
| 7b | Familial Hypercholesterolemia | Open-label | ≤6 active | 100 mg Day 1, 29 |
| 7c | Familial Hypercholesterolemia | Open-label | ≤6 active | 300 mg Day 1, 29 |
| 8 | TGs ≥300 mg/dl (3.39 mmol/L) | Open-label | ≤6 active | 200 mg Day 1, 29 |
| 9 | Familial Hypercholesterolemia | Open-label | ≤18 active | 200 mg Days 113, |
| | Extension Cohort (have completed | | | 197, 281, 365, 200 |
| | Cohort 7, 7b or 7c) | | | |

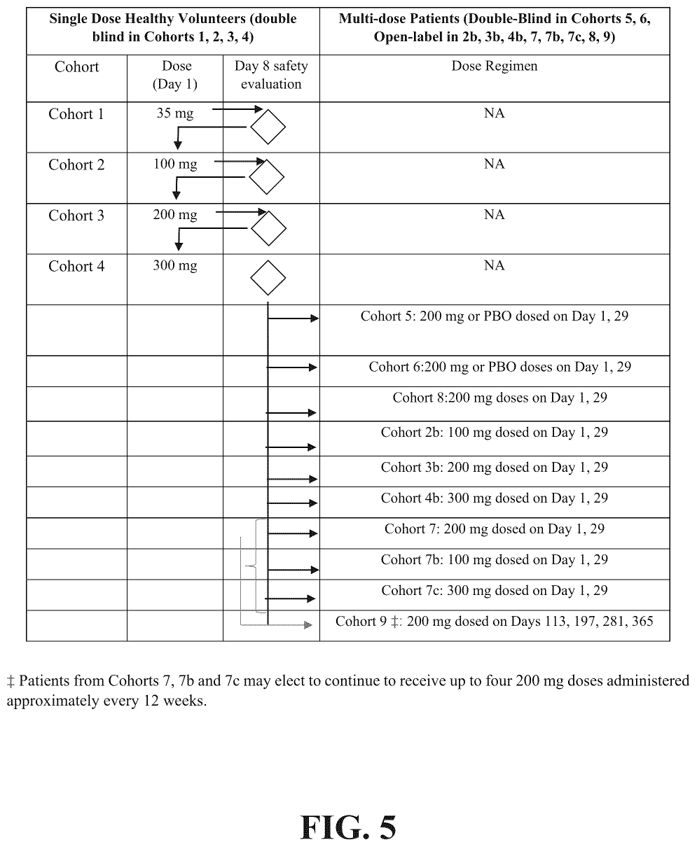

The number of subjects reflects the maximum number of up to 94 subjects that may be enrolled in the study (not including replacements). The dose escalation schedule schematic for the study is depicted in FIG. 5.

TABLE 7
Clinical Study Parameters

| | |
|---|---|
| Development Phase | Phase 1 |
| Study Objectives | Primary Objectives: |
| | To determine the incidence and frequency of adverse events |
| | possibly or probably related to treatment as a measure of the |
| | safety and tolerability of ANGPTL3 RNAi Drug Substance |
| | (ADS-003) using escalating single and multiple doses in |
| | healthy volunteers and multiple doses in dyslipidemic |
| | patients. |
| | Secondary Objectives: |
| | To evaluate the single-dose and multi-dose pharmacokinetics |
| | of ARO-ANG3 in healthy volunteers. |
| | To determine the reduction in fasting serum ANGPTL3 from |
| | baseline in response to a single and multiple doses of ARO- |
| | ANG3 as a measure of drug activity in healthy volunteers and |
| | in response to multiple doses of ARO-ANG3 in dyslipidemic |
| | patients (all values drawn after at least 8 hour fast). |
| | Exploratory Objectives: |
| | To evaluate the effect of single or multiple doses of ARO- |
| | ANG3 on change from baseline in fasting LDL-C, Total |
| | Cholesterol, non-HDL-C, HDL-C, VLDL-C, Triglycerides, |
| | Lp(a), apoB-48, apoB-100, apoC-III apoC-II, apoA-V, |
| | lipoprotein lipase mass (if feasible), hepatic lipase mass (if |
| | feasible), CETP mass (if feasible) and apoA-I (all values |
| | drawn after at least 8 hour fast). |
| | To evaluate the effect of single or multiple doses of ARO- |
| | ANG3 on changes from baseline in BMI. |
| | To evaluate the effect of single or multiple doses of ARO- |
| | ANG3 on changes from baseline in fasting serum blood |
| | glucose, C-peptide, hemoglobin A1C, GTT and fasting serum |
| | insulin. |
| | To evaluate the effect of multiple doses of ARO-ANG3 on |
| | change from baseline liver fat content using Magnetic |
| | Resonance Imaging (using MRI-PDFF) in Cohort 5 only. |
| | To evaluate the effect of multiple doses of ARO-ANG3 on |
| | change from baseline in post-prandial (post standardized high |
| | fat/high carbohydrate meal) serum TGs in specified cohorts. |
| | To evaluate excretion of ARO-ANG3 (full length and |
| | metabolites) and identify metabolites in plasma and urine in |
| | the multi-dose healthy volunteer cohorts. |
| Investigational | ANGPTL3 RNAi Drug Substance (ADS-003) (see Table 2), |
| Product (IP) | administered as Formulated ANGPTL3 RNAi Drug Substance |
| | (see Table 3) |
| Reference | Placebo (PBO): normal saline (0.9%) administered |
| Formulation | subcutaneously with matching volume. |
| Rationale for the | Treatment with ANGPTL3 RNAi Drug Substance (ADS-003) is |
| Study | anticipated to reduce hepatic production of ANGPTL3 via RNAi, |
| | leading to reductions in serum LDL-C, TG, VLDL-C and ApoB. |
| | The magnitude of the reduction and duration of effect will depend |
| | on the dose. Since to prior to this study there was no human |
| | clinical exposure to ANGPTL3 RNAi Drug Substance, an |
| | effective therapeutic dose to administer to patients with |
| | dyslipidemia is unknown, although in the RNAi field, non-human |
| | primate potency and duration is usually similar to that seen in |
| | humans. This study used a single-ascending-dose (SAD) in |
| | healthy volunteers to determine the dose required to reach and |
| | sustain maximal knockdown in ANGPTL3 serum levels, and the |
| | dose-response relationship in humans. In Cohort 5, ANGPTL3 |
| | RNAi Drug Substance was be evaluated in healthy volunteer |
| | subjects with hepatic steatosis as inhibition of intra-hepatic |
| | ANGPTL3 may lead to reduced intra-hepatic fat. Cohort 6 will |
| | evaluated the ability of ANGPTL3 inhibition to further reduce |
| | LDL-C levels in subjects already on lipid lowering therapy such |
| | as HMG-CoA-reductase inhibitors (“statins”). Cohorts 7 and 8 |
| | was intended to evaluate preliminary evidence of safety and drug |
| | activity in patient populations likely to benefit from ARO-ANG3 |
| | treatment. |
| | The rationale for addition of multiple dose NHV cohorts was to |
| | better understand multidose pharmacodynamic and |
| | pharmacokinetics at escalating doses. |
| | The rationale for addition of Cohorts 7b and 7c was to better |
| | understand dose response in patients with familial |
| | hypercholesterolemia. The proposed doses of 100 and 300 mg |
| | were evaluated previously in this same study in healthy |
| | volunteers. |
| | Cohort 9 was intended to allow HeFH patients enrolled in Cohorts |
| | 7, 76, 7c continued access to ANGPTL3 RNAi Drug Substance |
| | with the addition of four Q12 week doses. |

### Results

ANGPTL3 RNAi Drug Substance (ADS-003) was well tolerated without adverse changes in liver fat in steatotic subjects and no apparent adverse effects on liver transaminases. The duration of pharmacologic effect of ANGPTL3 RNAi Drug Substance was substantial with over three months of silencing of the gene target after a single dose.

In healthy volunteers, ARO-ANG3 produced reductions in ANGPTL3 (mean −45% to −78%) 12 weeks post-dose. Concurrent reductions in triglycerides (median −34% to −54%) and non-HDL-C (mean −18% to −29%) were observed with the 3 highest doses. Reduced LDL-C was seen with repeat dosing.

In the healthy volunteer single ascending dose (SAD) cohorts, 24 subjects received subcutaneously administered ANGPTL3 RNAi Drug Substance (ADS-003) (see Table 2), administered as Formulated ANGPTL3 RNAi Drug Substance (see Table 3) (35, 100, 200, or 300 mg) and 16 subjects received placebo on Day 1 (Cohorts 1-4). In the healthy volunteer multiple ascending dose (MAD) cohorts, 12 subjects received subcutaneously administered ANGPTL3 RNAi Drug Substance (ADS-003) (see Table 2), administered as Formulated ANGPTL3 RNAi Drug Substance (see Table 3) 100, 200, or 300 mg on Days 1 and 29. In the hepatic steatosis cohort, 6 subjects received repeat doses (200 mg) of subcutaneously administered ANGPTL3 RNAi Drug Substance (ADS-003) (see Table 2), administered as Formulated ANGPTL3 RNAi Drug Substance (see Table 3) and 3 subjects received placebo on days 1 and 29. Subjects in this cohort were required to have hepatic steatosis defined as a liver fat content of ≥10%, measured as the MRI-proton density fat fraction (MRI-PDFF).

Demographics and baseline characteristics for the health volunteer SAD cohorts (n=40, active treatment median age 46.0 years, 17 of 24 [70.8%] male: placebo median age 39.5 years, 12 of 16 [75.0%] male) and the health volunteer multiple ascending dose (MAD) cohort (n=12, active treatment median age 40.3 years, 8 of 12 [66.7%] male) are described in Table 6.

In the hepatic steatosis cohort (Cohort 5), mean (SD) percent change in ANGPTL3 from baseline at Day 113 was −85.3% (12.7%) compared to an increase of 13.0% (4.7%) for placebo. The median TG percent change from baseline at Day 113 was −44.1% compared to an increase of 47.1% for placebo. On Day 113, the non-HDL-C mean (SD) percent change from baseline was −36.7% (15.0%), compared to 0.2% (14.3%) in the placebo cohort. Concurrently, for VLDL-C, the mean (SD) percent change from baseline was −43.5% (20.8%) compared to 45.9% (50.8%) in the placebo cohort. The mean (SD) LDL-C percent change from baseline was −34.6% (14.1%) compared to −4.3% (29.3%) for placebo. The absolute change from baseline in liver fat at Day 71 ranged from −0.87% to −16.39% (mean absolute change of −4.35%) with relative changes from baseline ranging from −4.74% to −69.6% (mean relative change of −18.23%) in the active treatment group. In the placebo group at Day 71, absolute change in liver fat from baseline ranged from +3.68% to −4.89% (mean absolute change of −1.96%) with relative changes ranging from +20.91% to −29.32% (mean relative change of −8.56%). At Day 168 in the active treatment group, absolute changes from baseline in liver fat ranged from +2.58% to −12.09% (mean change of −5.1%). Relative changes ranged from +7.81% to −57.34% (mean relative change of −28.17%). In the placebo group, absolute change ranged from −2.84% to −6.35% (mean change of −4.25%), with relative changes ranging from −16.14% to −23.52% (mean change of −20.33%). Importantly, liver fat did not increase in a clinically significant manner in any single subject receiving ARO-ANG3 in this small cohort.

In this small sample of subjects with hepatic steatosis, ARO-ANG3 also decreased atherogenic lipoproteins and, importantly, repeat dosing did not increase liver fat, with most subjects showing a numerical decline in liver fat content. These results suggest that silencing ANGPTL3 protein synthesis with a hepatocyte-targeted RNAi agent (here, a hepatocyte-targeted double stranded small interfering RNA) is a viable approach for reducing residual cardiovascular associated with atherogenic lipoproteins. Additionally, in this small study Phase 1 study, ARO-ANG3 was not associated with an adverse aminotransferase or liver fat safety profile.

In Cohort 7, 200 mg of ARO-ANG3 demonstrated mean reductions in ANGPTL3 of 80% on Day 29 corresponding to mean nadir LDL-C reductions of 29% (mean absolute reductions of 47.7 md/dL) in patients with HeFH. This finding is highly clinically significant as Cohort 7 patients entered the study with mean LDL-C of 154 mg/dl (Range 100-181) despite the baseline use of statins and/or ezetimibe and in some cases, PCSK-9 inhibitors. This additional reduction in LDL-C with the addition of ANGPTL3 RNAi Drug Substance on top of maximal standard therapy could help these patients reach and maintain target LDL-C goals. Lower LDL-C is associated with reduced risk of atherosclerotic cardiovascular disease in the HeFH/HoFH population.

In summary, this early-stage study demonstrated that RNAi therapy targeting ANGPTL3 mRNA can safely and effectively lower circulating levels of atherogenic lipoproteins. ANGPTL3 RNAi Drug Substance showed greater potency and durability in TG-lowering effects and triglyceride-right lipoprotein (TRL)-lowering effects compared with current standard of care and has an advantage over fibrates and omega-3 fatty acids of lowering LDL-C. Overall, ANGPTL3 RNAi Drug Substance (ADS-003) was generally well tolerated when administered subcutaneously as a single dose in healthy volunteers and repeat doses in healthy volunteers and hepatic steatosis subjects. There were no deaths, life-threatening treatment-emergent adverse events (TEAEs), or TEAEs leading to drug discontinuation or premature withdrawal of any subject from the study. There were no treatment-related SAEs and the majority of TEAEs were either mild or moderate.

### Example 4. Phase 2 Clinical Trial to Evaluate the Efficacy and Safety of ANGPTL3 RNAi Drug Substance (ADS-003) in Subjects with Homozygous Familial Hypercholesterolemia

A Phase 2 study to evaluate the safety and efficacy of ANGPTL3 RNAi Drug Substance (ADS-003) in adults with homozygous familial hypercholesterolemia (HoFH) was initiated.

In this open-label Phase 2 study, 18 HoFH patients with a mean fasting baseline LDL-C of 10.0 mmol/L (range: 2.3 to 21.6 mmol/L) on lipid-lowering standard of care (including apheresis), were randomized to receive subcutaneous injections of either 200 mg or 300 mg of ANGPTL3 RNAi Drug Substance (ADS-003) (consisting of the Formulated ANGPTL3 RNAi Drug Substance as described in Table 3) on Day 1 and Week 12. The primary endpoint was fasting LDL-C percent change from baseline to Week 24. The initial cut-off date for the interim analysis was 17 Apr. 2023, and included 17 subjects who have completed Week 20.

At Week 20, the 200 mg dose of ANGPTL3 RNAi Drug Substance (ADS-003) (consisting of the Formulated ANGPTL3 RNAi Drug Substance as described in Table 3) achieved mean reductions from baseline (and mean percent change) in LDL-C(Martin Hopkins) of −4.4 mmol/L (48.1% reduction), ApoB of −1.0 g/L (39.2% reduction), and ANGPTL3 of −54.6 ng/mL (82.7% reduction).

At Week 20, the 300 mg dose of ANGPTL3 RNAi Drug Substance (ADS-003) (consisting of the Formulated ANGPTL3 RNAi Drug Substance as described in Table 3) achieved mean reductions from baseline (and mean percent change) in LDL-C(Martin Hopkins) of −5.0 mmol/L (44.0% reduction), ApoB of −0.9 g/L (34.5% reduction), and ANGPTL3 of −87.9 ng/ml (−80.1% reduction). Data are summarized in the following Table 8:

TABLE 8
Change from Baseline in Serum Lipid and Lipoprotein
Concentrations at Week 20 in the Phase 2 Clinical
Trial of Example 4 in HoFH Patients

| Serum Concentrations | |
|---|---|
| ANGPTL3 RNAi Drug Substance | |
| (ADS-003) (N = 15) | |
| 200 mg dose | 300 mg dose |
| (N = 8) | (N = 8) |

LDL-C (Martin- Hopkins)

| | | |
|---|---|---|
| Baseline for Week 20 | 9.7 (6.2) | 11.1 (5.7) |
| (mmol/L), Mean (SD) | (n = 8) | (n = 9) |
| Week 20 (mmol/L), Mean (SD) | 5.3 (3.9) | 6.1 (3.1) |
| | (n = 8) | (n = 9) |
| Change from Baseline to | −4.4 (2.9) | −5.0 (3.6) |
| Week 20 (mmol/L), Mean (SD) | (n = 8) | (n = 9) |
| Percent change from Baseline | −48.1 (13.3) | −44.0 (17.3) |
| to Week 20, Mean (SD) | (n = 8) | (n = 9) |

ApoB

| | | |
|---|---|---|
| Baseline for Week 20 (g/L), | 2.5 (1.4) | 2.6 (1.0) |
| Mean (SD) | (n = 8) | (n = 9) |
| Week 20 (g/L), Mean (SD) | 1.5 (0.9) | 1.7 (0.7) |
| | (n = 8) | (n = 9) |
| Change from Baseline to | −1.0 (0.7) | −0.9 (0.5) |
| Week 20 (g/L), Mean (SD) | (n = 8) | (n = 9) |
| Percent change from Baseline | −39.2 (12.6) | −34.5 (14.9) |
| to Week 20, Mean (SD) | (n = 8) | (n = 9) |

Abbreviations: ApoB = apolipoprotein B; LDL-C; low-density lipoprotein cholesterol; SD = standard deviation.
Notes:
As of the lipid data cut-off date (17 Apr. 2023) 17 of the 18 patients randomized in the study completed Week 20; N is the total number of patients per dosage group as of the data cut-off date; Baseline is the last measurement available prior to the first dose of study drug; n is the number of patients with measurements at both Baseline and at the Week 20 visit.

No drug discontinuations, drug-related SAEs or AEs related to elevated ALTs were reported. The most frequent AEs were injection site pain and erythema (n=2 (11.1%)), and nasopharyngitis (n=2 (11.1%)) and dizziness (n=2 (11.1%)). These interim data suggest that HoFH patients on lipid-lowering standard care who received either 200 mg or 300 mg of ANGPTL3 RNAi Drug Substance (ADS-003) (consisting of the Formulated ANGPTL3 RNAi Drug Substance as described in Table 3) administered by subcutaneous injection as an adjunct therapy to standard of case LDL-C lowering medications achieved additional reductions in LDL-C similar to the additional LDL-C reductions observed with ANGPTL3-targeted monoclonal antibodies; however, the currently marketed ANGPTL3-targeted monoclonal antibodies require IV infusion administration once monthly (administered via an IV infusion over 60 minutes), which may result in, among other things, potential patient compliance issues compared to a single subcutaneous injection administered quarterly.

Additional data reported through week 24 was consistent with the interim analysis, as shown in the following Table 8B:

TABLE 8B
Change from Baseline in Serum Lipid and Lipoprotein Concentrations at Week
20 and 24 in the Phase 2 Clinical Trial of Example 4 in HoFH Patients

| | Week 20 | | Week 24 | |
|---|---|---|---|---|
| | 200 mg | 300 mg | 200 mg | 300 mg |
| | ARO-ANG3 | ARO-ANG3 | ARO-ANG3 | ARO-ANG3 |
| | (N = 9) | (N = 9) | (N = 9) | (N = 9) |
| | Serum Concentrations | | | |
| Baseline | 372.1 (236.3) | 428.7 (220.4) | 342.7 (238.1) | 428.7 (220.4) |
| Mean (SD) | (n = 8) | (n = 9) | (n = 9) | (n = 9) |
| LDL-C, mg/dL | | | | |
| LDL-C Mean % | −46.9 (13.5) | −43.2 (17.1) | −36.8 (28.9) | −40.1 (18.3) |
| change (SD) | (n = 8) | (n = 9) | (n = 9) | (n = 9) |
| Baseline | 247.9 (137.4) | 256.3 (104.4) | 231.2 (138.0) | 256.3 (104.4) |
| Mean (SD) | (n = 8) | (n = 9) | (n = 9) | (n = 9) |
| ApoB, mg/dL | | | | |
| ApoB Mean % | −39.2 (12.6) | −34.5 (14.9) | −34.4 (24.5) | −32.3 (14.6) |
| change (SD) | (n = 8) | (n = 9) | (n = 9) | (n = 9) |
| Baseline | 403.6 (252.0) | 451.4 (220.2) | 372.9 (253.1) | 451.4 (220.2) |
| Mean (SD) | (n = 8) | (n = 9) | (n = 9) | (n = 9) |
| Non-HDL-C, | | | | |
| mg/dL | | | | |
| Non-HDL-C | −47.0 (12.4) | −43.1 (16.1) | −38.3 (27.2) | −40.3 (22.2) |
| Mean % change | (n = 8) | (n = 9) | (n = 9) | (n = 9) |
| (SD) | | | | |
| Baseline | 139.5 (91.4) | 103.9 (42.5) | 136.0 (86.1) | 103.9 (42.5) |
| Mean (SD) | (n = 8) | (n = 9) | (n = 9) | (n = 9) |
| TG, mg/dL | | | | |
| TG Mean % | −38.7 (29.8) | −28.1 (24.0) | −46.6 (24.5) | −30.9 (25.7) |
| change (SD) | (n = 8) | (n = 9) | (n = 9) | (n = 9) |
| Baseline | 157.6 (125.2) | 149.9 (116.5) | 187.4 (147.3) | 149.9 (116.5) |
| Mean (SD) | (n = 8) | (n = 9) | (n = 9) | (n = 9) |
| Lp(a), nmol/L | | | | |
| Lp(a) Mean % | −30.6 (22.8) | −22.4 (33.0) | −26.4 (22.6) | −30.6 (29.2) |
| change (SD) | (n = 8) | (n = 9) | (n = 9) | (n = 9) |

Abbreviations: ApoB = apolipoprotein B; LDL-C; low-density lipoprotein cholesterol; (Friedewald); non-HDL-C = non-high density lipoprotein cholesterol; TG = triglyceride; Lp(a) = lipoprotein (a); SD = standard deviation.
Notes:
N is the total number of patients per dosage group; Baseline is the last measurement available prior to the first dose of study drug; n is the number of patients with measurements at both Baseline and at the indicated visit.
Data cutoff date (16 May 2023), Source table 14.2.1.1

As shown in the Tables above, 200 mg and 300 mg ARO-ANG3 produced significant reduction of LDL-C (up to −46.9%) and additional atherogenic lipoproteins, including TGs, non-HDL-C, apolipoprotein B and lipoprotein (a) by Week 4 with sustained reduction following the second injection to Week 24 (−22.4% to −47.0%). The LDL-C reduction is similar to intravenously administered commercially available ANGPTL3-targeted monoclonal antibodies, with less frequent subcutaneous dose administration. No drug discontinuation, drug-related SAEs or AEs related to elevated ALTs were reported. In a total of 18 subjects, TEAEs were reported for 10 subjects. Notably, the injection site AEs were pain (n=2 (11.1%)) and erythema (n=1 (5.6%)) and were mild and resolved rapidly.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating an ANGPTL3-related disease or disorder in a human subject in need thereof, the method comprising administering to the subject a pharmaceutical composition that comprises ANGPTL3 RNAi Drug Substance preferably in a pharmaceutically acceptable salt form, the ANGPTL3 RNAi Drug Substance comprising an antisense strand wherein the nucleotides starting from the 5′ end of the antisense strand comprise the nucleotide sequence (5′→3′): UACUGAUCAAAUAUGUUGAGC (SEQ ID NO: 3) differing by 0, 1, or 2 nucleotides, and a sense strand comprising the nucleotide sequence (5′→3′): GCUCAACAUAUUUGAUCAGUA (SEQ ID NO:5) differing by 0, 1, or 2 nucleotides, wherein the ANGPTL3 RNAi Drug Substance is administered to the subject at a dose of at least 50 mg of the ANGPTL3 RNAi Drug Substance.

2. The method of claim 1, wherein the sense strand of the ANGPTL3 RNAi Drug Substance is conjugated to a targeting ligand that comprises N-acetyl-galactosamine.

3. The method of claim 1 or claim 2, wherein the ANGPTL3 RNAi Drug Substance comprises an antisense strand comprising the nucleotide sequence: usAfscsUfgAfuCfaAfaUfaUfgUfuGfaGfsc (SEQ ID NO:2), and a sense strand comprising the nucleotide sequence: (NAG37)s(invAb)sgcucaacaUfAfUfuugaucaguas(invAb) (SEQ ID NO:6), wherein a represents 2′-O-methyl adenosine, c represents 2′-O-methyl cytidine, g represents 2′-O-methyl guanosine, and u represents 2′-O-methyl adenosine: Af represents 2′-fluoro adenosine, Cf represents 2′-fluoro cytidine, Gf represents 2′-fluoro guanosine, and Uf represents 2′-fluoro uridine: s represents a phosphorothioate linkage: (invAb) represents an inverted abasic deoxyribose residue; and (NAG37)s comprises the structure represented by: 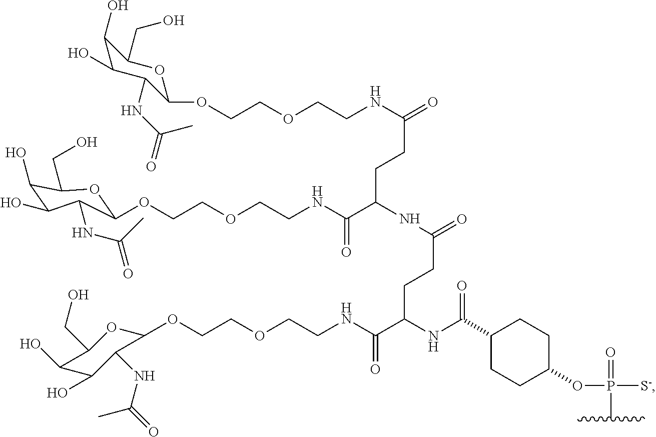
wherein the ANGPTL3 RNAi Drug Substance is administered a dose of between about 50 mg to about 400 mg of the ANGPTL3 RNAi Drug Substance.

4. The method of any one of claims 1-3, wherein the ANGPTL3 RNAi Drug Substance is administered by subcutaneous injection.

5. The method of any one of claims 1-4, wherein the method further comprises administering a second dose of the pharmaceutical composition comprising between 50 mg to about 400 mg of the ANGPTL3 RNAi Drug Substance:
a. about one month after the first dose; or
b. about 12 weeks (q12w) after the first dose; or
c. about four months after the first dose; or
d. about six months (q6m) after the first dose;
wherein the first dose and the second dose are administered by subcutaneous injection.

6. The method of any one of claims 1-5, further comprising administering additional doses after the second dose, wherein the additional doses are administered about one month apart.

7. The method of any one of claims 1-5, further comprising administering additional doses after the second dose, wherein the additional doses are administered about 12 weeks apart (q12w).

8. The method of any one of claims 1-5, further comprising administering additional doses after the second dose, wherein the additional doses are administered about four months apart.

9. The method of any one of claims 1-5, further comprising administering additional doses after the second dose, wherein the additional doses are administered about six months apart (q6m).

10. The method of any one of claims 1-9, wherein each dose is between about 50 mg to about 400 mg of the ANGPTL3 RNAi Drug Substance.

11. The method of any one of claims 1-9, wherein each dose is between about 100 mg to about 300 mg of the ANGPTL3 RNAi Drug Substance.

12. The method of any one of claims 1-9, wherein each dose is between about 200 mg to about 300 mg of the ANGPTL3 RNAi Drug Substance.

13. The method of any one of claims 1-9, wherein each dose of the ANGPTL3 RNAi Drug Substance is about 50 mg.

14. The method of any one of claims 1-9, wherein each dose of the ANGPTL3 RNAi Drug Substance is about 100 mg.

15. The method of any one of claims 1-9, wherein each dose of the ANGPTL3 RNAi Drug Substance is about 200 mg.

16. The method of any one of claims 1-9, wherein each dose of the ANGPTL3 RNAi Drug Substance is about 300 mg.

17. The method of any one of claims 1-9, wherein each dose of the ANGPTL3 RNAi Drug Substance is about 400 mg.

18. The method of any one of claims 1-17, wherein administration of the ANGPTL3 RNAi Drug Substance does not cause the subject to exhibit a significant increase in liver fat.

19. The method of claim 18, wherein the subject has a reduction in liver fat subsequent to administration of the ANGPTL3 RNAi Drug Substance.

20. The method of any one of claims 1-19, wherein administration of the ANGPTL3 RNAi Drug Substance does not cause the subject to exhibit a significant increase in alanine aminotransferase (ALT).

21. The method of any one of claims 1-20, wherein administration of the ANGPTL3 RNAi Drug Substance does not cause the subject to exhibit a significant increase in aspartate aminotransferase (AST).

22. The method of any one of claims 1-21, wherein the subject is further administered an additional therapeutic for the treatment of an ANGPTL3-related disease or disorder.

23. The method of any one of claims 1-22, wherein the ANGPTL3-related disease or disorder is hypertriglyceridemia (including severe hypertriglyceridemia (SHTG)), homozygous familial hypercholesterolemia (HoFH), hypertriglyceridemia induced pancreatitis, metabolic syndrome, obesity, hyperlipidemia, mixed dyslipidemia, abnormal lipid and/or cholesterol metabolism, atherosclerosis, cardiovascular disease, atherosclerotic cardiovascular disease (ASCVD), type II diabetes mellitus, coronary artery disease, non-alcoholic steatohepatitis, non-alcoholic fatty liver disease, heterozygous familial hypercholesterolemia (HeFH), statin resistant hypercholesterolemia, other dyslipidemias, and other metabolic-related disorders and diseases.

24. The method of any one of claims 1-22, wherein the ANGPTL3-related disease or disorder is a dyslipidemia.

25. The method of any one of claims 1-22, wherein the ANGPTL3-related disease or disorder is mixed dyslipidemia.

26. The method of any one of claims 1-22, wherein the ANGPTL3-related disease or disorder is atherosclerotic cardiovascular disease (ASCVD).

27. The method of any one of claims 1-22, wherein the ANGPTL3-related disease or disorder is hypertriglyceridemia, either with or without a history of pancreatitis.

28. The method of any one of claims 1-22, wherein the ANGPTL3-related disease or disorder is severe hypertriglyceridemia (SHTG), either with or without a history of pancreatitis.

29. The method of any one of claims 1-22, wherein the ANGPTL3-related disease or disorder is familial hypercholesterolemia.

30. The method of any one of claims 1-22, wherein the ANGPTL3-related disease or disorder is homozygous familial hypercholesterolemia (HoFH).

31. The method of any one of claims 1-22, wherein the ANGPTL3-related disease or disorder is heterozygous familial hypercholesterolemia (HeFH).

32. The method of any one of claims 1-22, wherein the subject has been previously diagnosed with hepatic steatosis.

33. The method of any one of claims 1-32, wherein the pharmaceutical composition is packaged in a kit, container, pack, dispenser, pre-filled syringe, or vials.

34. The method of any one of claims 1-33, wherein the pharmaceutical composition comprises, consists of, or consists essentially of the Formulated ANGPTL3 RNAi Drug Substance described in Table 3 comprised of the ANGPTL3 RNAi Drug Substance in a concentration of about 200 mg/mL, sodium phosphate monobasic monohydrate in a concentration of about 0.061 mg/mL, anhydrous sodium phosphate dibasic in a concentration of about 0.062 mg/mL, and water in a concentration of about 879.2 mg/mL.

35. The method of any one of claims 1-34, wherein the administration of one or more doses of the pharmaceutical composition is performed by the subject.

36. The method of any one of claims 1-34, wherein the administration of one or more doses of the pharmaceutical composition is performed by a medical professional.

37. The method of any one of claims 1-36, wherein the ANGPTL3 protein levels are reduced by greater than 50% from baseline levels in the human subject.

38. The method of any one of claims 1-37, wherein the serum triglyceride (TG) levels are reduced by greater than 40% from baseline levels in the human subject.

39. The method of claim 38, wherein the serum TG levels are reduced by greater than 50% from baseline levels in the human subject.

40. The method of any one of claims 1-39, wherein one or more of serum low density lipoprotein cholesterol (LDL-C) levels, serum non-high density lipoprotein cholesterol (non-HDL-C) levels, serum remnant cholesterol levels, and apolipoprotein B levels are reduced in the human subject compared to its respective baseline levels.

41. The method of any one of claims 1-40, wherein the subject has fasting or post-prandial baseline triglyceride levels greater than 150 mg/dL.

42. The method of any one of claims 1-41, wherein the subject has fasting or post-prandial baseline triglyceride levels greater than 500 mg/dL.

43. The method of any one of claims 1-42, wherein the ANGPTL3 RNAi Drug Substance is administered as a pharmaceutically acceptable salt, pharmaceutically acceptable mixed salt, free acid, or a combination thereof.

44. The method of claim 43, wherein the ANGPTL3 RNAi Drug Substance is administered as a pharmaceutically acceptable sodium salt.

45. A unit dosage form comprising ANGPTL3 RNAi Drug Substance preferably in a pharmaceutically acceptable salt form, wherein the ANGPTL3 RNAi Drug Substance comprises an antisense strand comprising the nucleotide sequence: usAfscsUfgAfuCfaAfaUfaUfgUfuGfaGfsc (SEQ ID NO:2), and a sense strand comprising the nucleotide sequence: (NAG37)s(invAb)sgcucaacaUfAfUfuugaucaguas(invAb) (SEQ ID NO:6), wherein a represents 2′-O-methyl adenosine, c represents 2′-O-methyl cytidine, g represents 2′-O-methyl guanosine, and u represents 2′-O-methyl adenosine; Af represents 2′-fluoro adenosine, Cf represents 2′-fluoro cytidine, Gf represents 2′-fluoro guanosine, and Uf represents 2′-fluoro uridine; s represents a phosphorothioate linkage; (invAb) represents an inverted abasic deoxyribose residue; and (NAG37) s comprises the structure represented by: 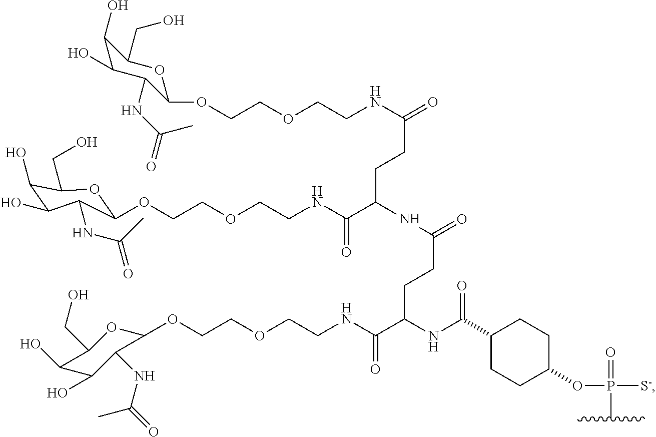
at an amount of between about 50 mg to about 400 mg of the ANGPTL3 RNAi Drug Substance.

46. A pharmaceutical composition for use in treating an ANGPTL3-related disease or disorder in a human subject in need thereof, wherein said use comprises the administration of the pharmaceutical composition comprising an ANGPTL3 RNAi agent, wherein the pharmaceutical composition is administered to the subject at a first dose of between about 50 mg to about 400 mg of the ANGPTL3 RNAi agent, and wherein a second dose of the pharmaceutical composition is administered to the subject at a dose of between about 50 mg to about 400 mg of the ANGPTL3 RNAi agent, wherein the second dose is: (i) about one month after the first dose; (ii) about 12 weeks after the first dose (q12w or q3m); (iii) about 4 months after the first dose; or (iv) about 6 months after the first dose (q6m), and wherein the first dose and the second dose are administered by subcutaneous injection.

47. The pharmaceutical composition of claim 46 for use in treating an ANGPTL3-related disease or disorder in a human subject in need thereof, wherein said use comprises the administration of the pharmaceutical composition comprising an ANGPTL3 RNAi agent, wherein:
a. the pharmaceutical composition is administered to the human subject at a first dose of between about 200 mg to about 300 mg of the ANGPTL3 RNAi agent,
b. the pharmaceutical composition is administered to the human subject at a second dose of between about 200 mg to about 300 mg of the ANGPTL3 RNAi agent between about one month to about six months after the first dose, and
c. the pharmaceutical composition is administered to the human subject at a third dose of between about 200 mg to about 300 mg of the ANGPTL3 RNAi agent between about one month to about six months after the second dose.

48. The pharmaceutical composition of claim 46 or 47 for use in treating an ANGPTL3-related disease or disorder in a human subject in need thereof, wherein the pharmaceutical composition comprises an ANGPTL3 RNAi agent that is a pharmaceutically acceptable sodium salt, wherein the ANGPTL3 RNAi agent comprises an antisense strand comprising the nucleotide sequence: usAfscsUfgAfuCfaAfaUfaUfgUfuGfaGfsc (SEQ ID NO: 2), and a sense strand comprising the nucleotide sequence: (NAG37)s(invAb)sgcucaacaUfAfUfuugaucaguas(invAb) (SEQ ID NO:6),
wherein a represents 2′-O-methyl adenosine, c represents 2′-O-methyl cytidine, g represents 2′-O-methyl guanosine, and u represents 2′-O-methyl adenosine; Af represents 2′-fluoro adenosine, Cf represents 2′-fluoro cytidine, Gf represents 2′-fluoro guanosine, and Uf represents 2′-fluoro uridine; s represents a phosphorothioate linkage; (invAb) represents an inverted abasic deoxyribose residue; and (NAG37) s comprises the structure represented by: 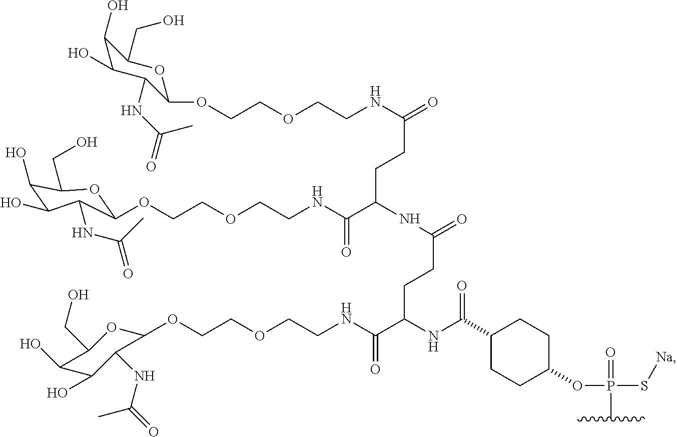
wherein the pharmaceutical composition does not cause significant increases in liver fat measured as the MRI-proton density fat fraction (MRI-PDFF), when administered to the subject.

49. The pharmaceutical composition of any one of claims claim 46-48, wherein the pharmaceutical composition does not cause significant increases in AST or ALT liver enzymes when administered to the subject.


SEQUENCE LISTING (6 sequences)

SEQ ID NO: 1 - RNA, 2926 nt, Homo sapiens
  source 1..2926: mol_type = mRNA
agaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatttgttaaaatatatactgtatacattttatatattttaacacttaatactatgaaaacaaataattgtaaaggaatcttgtcagattacagtaagaatgaacatatttgtggcatcgagttaaagtttatatttcccctaaatatgctgtgattctaatacattcgtgtaggttttcaagtagaaataaacctcgtaacaagttactgaacgtttaaacagcctgacaagcatgtatatatgtttaaaattcaataaacaaagacccagtccctaaattatagaaatttaaattattcttgcatgtttatcgacatcacaacagatccctaaatccctaaatccctaaagattagatacaaattttttaccacagtatcacttgtcagaatttatttttaaatatgattttttaaaactgccagtaagaaattttaaattaaacccatttgttaaaggatatagtgcccaagttatatggtgacctacctttgtcaatacttagcattatgtatttcaaattatccaatatacatgtcatatatatttttatatgtcacatatataaaagatatgtatgatctatgtgaatcctaagtaaatattttgttccagaaaagtacaaaataataaaggtaaaaataatctataattttcaggaccacagactaagctgtcgaaattaacgctgatttttttagggccagaataccaaaatggctcctctcttcccccaaaattggacaatttcaaatgcaaaataattcattatttaatatatgagttgcttcctctatttggtttcc

SEQ ID NO: 2 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = cm
  modified_base 3^4: mod_base = OTHER; note = phosphorothioate linkage
  modified_base 4: mod_base = OTHER; note = 2'-fluorouridine
  modified_base 5: mod_base = gm
  modified_base 6: mod_base = OTHER; note = 2'-fluoroadenosine
  modified_base 7: mod_base = um
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = um
  modified_base 18: mod_base = OTHER; note = 2'-fluoroguanosine
  modified_base 19: mod_base = OTHER; note = 2'-O-methyladenosine
  modified_base 20: mod_base = OTHER; note = 2'-fluoroguanosine
  modified_base 20^21: mod_base = OTHER; note = phosphorothioate linkage
  modified_base 21: mod_base = cm
  source 1..21: mol_type = other RNA
tactgatcaaatatgttgagc

SEQ ID NO: 3 - RNA, 21 nt, synthetic construct
  source 1..21: mol_type = other RNA
tactgatcaaatatgttgagc

SEQ ID NO: 4 - RNA, 21 nt, synthetic construct
  modified_base 1: mod_base = gm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluorouridine
  modified_base 12: mod_base = um
  modified_base 13: mod_base = um
  modified_base 14: mod_base = gm
  modified_base 15: mod_base = OTHER; note = 2'-O-methyladenosine
  modified_base 16: mod_base = um
  modified_base 17: mod_base = cm
  modified_base 18: mod_base = OTHER; note = 2'-O-methyladenosine
  modified_base 19: mod_base = gm
  modified_base 20: mod_base = um
  modified_base 21: mod_base = OTHER; note = 2'-O-methyladenosine
  source 1..21: mol_type = other RNA
gctcaacatatttgatcagta

SEQ ID NO: 5 - RNA, 21 nt, synthetic construct
  source 1..21: mol_type = other RNA
gctcaacatatttgatcagta

SEQ ID NO: 6 - RNA, 21 nt, synthetic construct
  modified_base 1: mod_base = gm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluorouridine
  modified_base 12: mod_base = um
  modified_base 13: mod_base = um
  modified_base 14: mod_base = gm
  modified_base 15: mod_base = OTHER; note = 2'-O-methyladenosine
  modified_base 16: mod_base = um
  modified_base 17: mod_base = cm
  modified_base 18: mod_base = OTHER; note = 2'-O-methyladenosine
  modified_base 19: mod_base = gm
  modified_base 20: mod_base = um
  modified_base 21: mod_base = OTHER; note = 2'-O-methyladenosine
  source 1..21: mol_type = other RNA
gctcaacatatttgatcagta